(19) United States
(12) Patent Application Publication
(10) Pub. No.: US 2007/0088067 A1
(43) Pub. Date: Apr. 19, 2007

(54) Novel amidine compounds for treating microbial infections

(76) Inventors: Richards R. Tidwell, Pittsboro, NC (US); David W. Boykin, Atlanta, GA (US); Reto Brun, Basel (CH); Chad E. Stephens, Villa Rica, GA (US); Arvind Kumar, Lilburn, GA (US)

Correspondence Address: JENKINS, WILSON, TAYLOR & HUNT, P. A., 3100 TOWER BLVD, SUITE 1200, DURHAM, NC, 27707, US

(21) Appl. No.: 10/570,584

(22) PCT Filed: Sep. 5, 2003
(86) PCT No.: PCT/US03/27963

§ 371 Date: Nov. 17, 2006

(51) Int. Cl. A61K 31/42; C07D 261/06; A61K 31/155; C07C 257/18

(52) U.S. Cl. 514/378; 514/637; 548/247; 564/243; 564/244

(57) ABSTRACT

Novel amidine and diamidine compounds are useful in the treatment of microbial infections, including mycobacterial, fungal and protozoal infections. Pharmaceutical formulations comprising these compounds can be used in methods of treating microbial infections.

### TECHNICAL FIELD

The presently disclosed subject matter relates to novel amidine compounds useful for treating microbial infections. More particularly, the presently disclosed subject matter relates to mono- and diamidine compounds useful for treating microbial infections, including mycobacterial, fungal and protozoal infections.

### ABBREVIATIONS

δ=chemical shift
Ac=acetyl
AcO=acetoxy
AcOH=acetic acid
Ac₂O=acetic anhydride
Bu=butyl
° C.=degrees Celsius
calcd=calculated
cm=centimeters
dec=decomposition point
DMF=dimethylformamide
DMSO=dimethylsulfoxide
EtOAc=ethyl acetate
EtOH=ethanol
FAB=fast atom bombardment
g=grams
h=hours
HPLC=high-pressure liquid chromatography
Hz=hertz
kg=kilograms
KO-t-Bu=potassium tert-butoxide
L. d.=Leishmania donovani
M=molar
Me=methyl
MeO=methoxy
MHz=megahertz
mL=milliliters
mm=millimeters
mM=millimolar
m.p.=melting point
MS=mass spectroscopy
NBS=N-bromosuccinimide
NH₂OH.HCl=hydroxylamine hydrochloride
NMR=nuclear magnetic resonance
Pd/C=10% palladium on carbon
P. f.=Plasmodium falciparum
psi=pounds per square inch
T. br.=Trypanosoma brucei rhodesiense
THF=tetrahydrofuran
TLC=thin-layer chromatography
TMS=trimethylsilyl
UV=ultraviolet

### BACKGROUND ART

The incidence of microbial infections (e.g., mycobacterial, fungal and protozoal infections) in the immunocompromised population has significantly increased over the past several years. In particular, Candida species, especially Candida albicans, are often significant pathogens in patients infected with human immunodeficiency virus (HIV). Another pathogen, Pneumocystis carinii, causes a form of pneumonia (PCP) that is believed to be one of the leading causes of death in patients suffering from AIDS.

Human African trypanosomiasis (HAT) has reemerged as a threat to over 60 million people. Current estimates are that between 350,000 and 450,000 people are infected.

Other severe and life-threatening microbial infections are caused by Mycobacterium tuberculosis, Aspergillus spp., Cryptosporidium parvum, Giardia lamblia, Plasmodium spp., Toxoplasma gondii, Fusarium solani, and Cryptococcus neoformans.

The antimicrobial properties of dicationic molecules have been studied since the 1930's. Compounds of this type have typically utilized amidine groups as the cationic moieties, and their activities against a number of pathogens including Cryptosporidium parvum, Giardia lamblia, Leishmania spp., Plasmodium spp., Pneumocystis carinii, Toxoplasma gondii, Trypanosoma spp., Candida albicans, Aspergillus spp. and Cryptococcus neoformans have been reported. See e.g., King, H. et al., Ann. Trop. Med. Parasitol. 1938, 32, 177-192; Blagburn, B. L. et al., Antimicrob. Agents Chemother. 1991, 35, 1520- 1523; Bell, C. A. et al., Antimicrob. Agents Chemother. 1991, 35, 1099-1107; Bell, et al., Antimicrob. Agents Chemother. 1990, 34, 1381-1386; Kirk, R. et al., Ann. Trop. Med. Parastiol. 1940, 34,181-197; Fulton, J. D. Ann. Trop. Med. Parasitol. 1940, 34, 53-66; Ivady, V. G. et al., Monatschr. Kinderheilkd. 1958, 106, 10-14; Boykin, D. W. et al., . J. Med. Chem. 1995, 38, 912-916; Boykin, D. W. et al., J. Med. Chem. 1998, 41, 124-129; Francesconi et al., J. Med. Chem. 1999, 42, 2260-2265; Lindsay, D. S. et al., Antimicrob. Agents Chemother. 1991, 35, 1914-1916; Lourie, E. M; et al., Ann. Trop. Med. Parasitol. 1939, 33, 289-304; Lourie, E. M. et al., Ann. Trop. Med. Parasitol. 1939, 33, 305-312; Das, B. P. et al., J Med. Chem. 1976, 20, 531-536; Del Poeta, M. et al., J. Antimicrob. Chemother. 1999, 44, 223-228; Del Poeta, M. et al., Antimicrob. Agents Chemother. 1998, 42, 2495-2502; Del Poeta, M. et al., Antimicrob. Agents Chemother. 1998, 42, 2503-2510.

Despite the broad range of activity exhibited by diamidines, only one compound of this chemical type, pentamidine, has seen significant clinical use. Pentamidine has been used clinically against African trypanosomiasis, antimony-resistant leishmaniasis, and P. carinii pneumonia. See e.g., Apted, F. I. C., Pharmacol. Ther. 1980, 11, 391-413; Bryceson, A. D. M. et al., Trans. Roy. Soc. Trop. Med. Hyg. 1985, 79, 705-714; Hughes, W. T.; et al., Antimicrob. Agents Chemother. 1974, 5, 289-293.

Thus, there continues to be a need for improvement in the art for additional compounds having desirable anti-microbial activity, whether against the representative pathogens referenced above or against other pathogens.

### SUMMARY

The presently disclosed subject matter relates to the use of amidine compounds in the treatment of microbial infections, including fungal infections. In particular, the disclosed subject matter relates to a method of treating or preventing a microbial infection in a subject comprising administering to the subject a therapeutic amount of an amidine compound. Among the compounds for use in the disclosed subject matter are those according to Formula I-VI, such that, when administered, microbial infections are reduced or inhibited.

A first aspect of the presently disclosed subject matter is a compound of Formula (I): 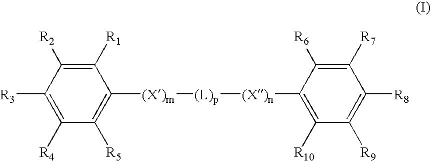
wherein:

X′ and X″ are each independently selected from the group consisting of alkyl, alkylene, oxygen, oxy, oxyalkyl, alkyloxy, alkyloxyalkyl, and

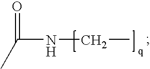

m, n, p, and q are each independently an integer from 0 to 10;

L is selected from the group consisting of hydroxyalkyl, 1,2-oxazole, 1,3-oxazole, phenyl, naphthyl, pyrimidine, alkyl-substituted pyrimidine and

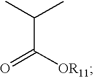

wherein R₁₁ is H or alkyl;

R₁, R₂, R₃, R₄, R₅, R₆, R₇, R₈, R₉, and R₁₀ are each independently selected from the group consisting of H, alkyl, hydroxyl, oxyalkyl, alkyloxy, halo, aryl, and Y, wherein at least one of R₁, R₂, R₃, R₄, R₅, R₆, R₇, R₈, R₉, and R₁₀ is Y, and Y is selected from the group consisting of:

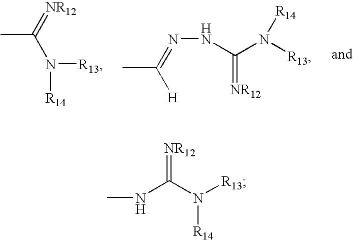

wherein:

R₁₂ is selected from the group consisting of H, hydroxyl, cycloalkyl, aryl, aralkyl, alkoxyl, hydroxycycloalkyl, alkoxycycloalkyl, hydroxyalkyl, aminoalkyl, acyloxy, and alkylaminoalkyl;

R₁₃ and R₁₄ are each independently selected from the group consisting of H, hydroxyl, alkyl, alkoxyalkyl, cycloalkyl, aryl, aralkyl, hydroxyalkyl, aminoalkyl, and alkylaminoalkyl;

or R₁₂ and R₁₃ together represent a C₂ to C₁₀ alkyl, hydroxyalkyl, or alkylene;

or R₁₂ and R₁₃ together are:

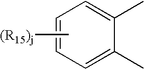

wherein:

j is an integer from 1 to 3, and R₁₅ is H or Y, as set forth above.

A second aspect of the presently disclosed subject matter is a compound of Formula (II): 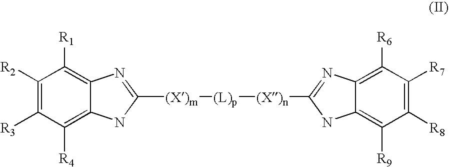
wherein:

m is an integer from 1 to 5;

n is an integer from 0 to 5;

p is an integer from 0 to 5;

X′ and X″ are each independently phenyl or thiophene;

L is selected from the group consisting of C₁₋₁₀ straight chain alkyl, C₁₋₁₀ branched chain alkyl, cycloalkyl, phenyl; and alkyl-substituted phenyl;

R₁, R₂, R₃, R₄, R₅, R₆, R₇, R₈, and R₉ are each independently selected from the group consisting of H, alkyl, hydroxyl, alkyloxy, oxyalkyl, halo, aryl, and Y, wherein at least one of R₁, R₂, R₃, R₄, R₅, R₆, R₇, R₈, and R₉ is Y, and Y is selected from the group consisting of:

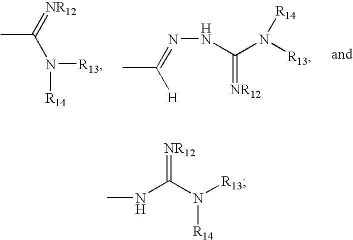

wherein:

R₁₂ is selected from the group consisting of H, hydroxyl, cycloalkyl, aryl, aralkyl, alkoxyl, hydroxycycloalkyl, alkoxycycloalkyl, hydroxyalkyl, aminoalkyl, acyloxyl, and alkylaminoalkyl;

R₁₃ and R₁₄ are each independently selected from the group consisting of H, hydroxyl, alkyl, alkoxyalkyl, cycloalkyl, aryl, aralkyl, hydroxyalkyl, aminoalkyl, and alkylaminoalkyl;

or R₁₂ and R₁₃together represent a C₂ to C₁₀ alkyl, hydroxyalkyl, or alkylene;

or R₁₂ and R₁₃ together are:

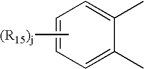

wherein:

j is an integer from 1 to 3, and R₁₅ is H or Y, as set forth above.

A third aspect of the presently disclosed subject matter is a compound of Formula (III): 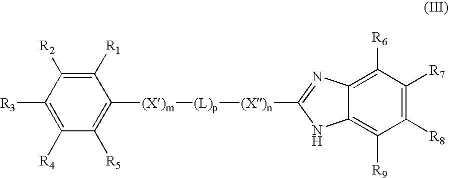
wherein:

L is phenyl, pyridine, or hydroxy-phenyl;

m and n are each independently an integer from 0 to 5;

X′ and X″ are each independently selected from the group consisting of C₁₋₁₀ straight chain alkyl, C₁₋₁₀ branched chain alkyl, and cycloalkyl;

R₁, R₂, R₃, R₄, R₅, R₆, R₇, R₈, and R₉ are each independently selected from the group consisting of H, alkyl, hydroxyl, alkyloxy, oxyalkyl, halo, aryl, and Y, wherein at least one of R₁, R₂, R₃, R₄, R₅, R₆, R₇, R₈, and R₉ is Y, and Y is selected from the group consisting of:

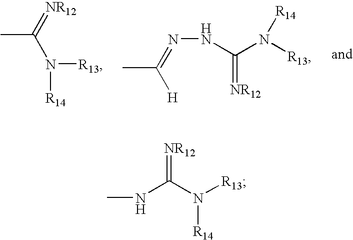

wherein:

R₁₂ is selected from the group consisting of H, hydroxyl, cycloalkyl, aryl, aralkyl, alkoxyl, hydroxycycloalkyl, alkoxycycloalkyl, hydroxyalkyl, aminoalkyl, acyloxyl, and alkylaminoalkyl;

R₁₃ and R₁₄ are each independently selected from the group consisting of H, hydroxyl, alkyl, alkoxyalkyl, cycloalkyl, aryl, aralkyl, hydroxyalkyl, aminoalkyl, and alkylaminoalkyl;

or R₁₂ and R₁₃ together represent a C₂ to C₁₀ alkyl, hydroxyalkyl, or alkylene;

or R₁₂ and R₁₃ together are:

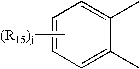

wherein:

j is an integer from 1 to 3, and R₁₅ is H or Y, as set forth above.

A fourth aspect of the presently disclosed subject matter is a compound of Formula (IV):

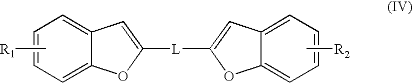

wherein L is selected from the group consisting of C₂₋₁₀ straight chain alkyl, C₁₋₁₀ branched chain alkyl, and cycloalkyl;

R₁ and R₂ are selected from the group consisting of:

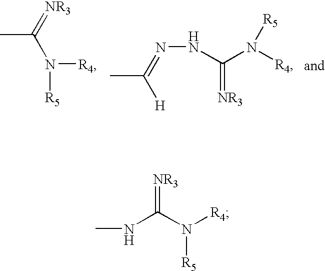

wherein R₃ is selected from the group consisting of H, hydroxyl, cycloalkyl, aryl, aralkyl, alkoxyl, hydroxycycloalkyl, alkoxycycloalkyl, hydroxyalkyl, aminoalkyl, acyloxyl, and alkylaminoalkyl;

R₄ and R₅ are each independently selected from the group consisting of H, hydroxyl, alkyl, alkoxyalkyl, cycloalkyl, aryl, aralkyl, hydroxyalkyl, aminoalkyl, and alkylaminoalkyl;

or R₃ and R₄together represent a C₂ to C₁₀ alkyl, hydroxyalkyl, or alkylene;

or R₄ and R₅ together are:

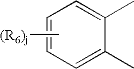

wherein:

j is a number from 1 to 3, and R₆ is selected from the group consisting of H and the groups from which R₁ and R₂ may be selected.

A fifth aspect of the presently disclosed subject matter is a compound of Formula (V):

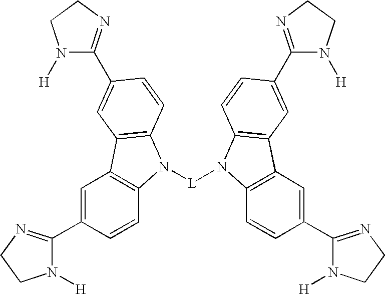

wherein L is an alkyl.

A sixth aspect of the presently disclosed subject matter is a compound of Formula (VI):

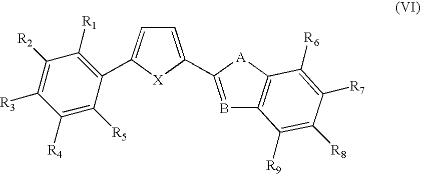

wherein:

X is oxygen;

A and B are each independently either nitrogen or oxygen;

R₁, R₂, R₃, R₄, R₅, R₆, R₇, R₈, and R₉ are each independently selected from the group consisting of H, alkyl, hydroxyl, alkyloxy, oxyalkyl, halo, aryl, and Y, wherein at least one of R₁, R₂, R₃, R₄, R₅, R₆, R₇, R₈, and R₉ is Y, and Y is selected from the group consisting of:

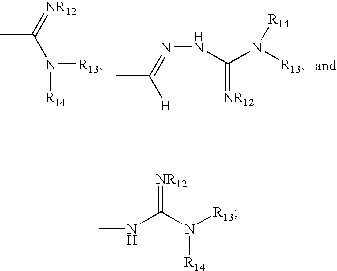

wherein:

R₁₂ is selected from the group consisting of H, hydroxyl, cycloalkyl, aryl, aralkyl, alkoxyl, hydroxycycloalkyl, alkoxycycloalkyl, hydroxyalkyl, aminoalkyl, acyloxyl, and alkylaminoalkyl;

R₁₃ and R₁₄ are each independently selected from the group consisting of H, hydroxyl, alkyl, alkoxyalkyl, cycloalkyl, aryl, aralkyl, hydroxyalkyl, aminoalkyl, and alkylaminoalkyl;

or R₁₂ and R₁₃ together represent a C₂ to C₁₀ alkyl, hydroxyalkyl, or alkylene;

or R₁₂ and R₁₃ together are:

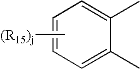

wherein:

j is an integer from 1 to 3, and R₁₅ is H or Y, as set forth above.

A seventh aspect of the presently disclosed subject matter is a compound of Formula (VII): 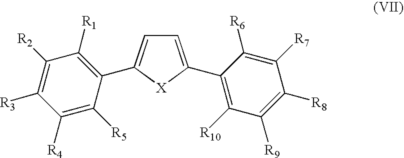
wherein:

X is oxygen; and

R₁, R₂, R₃, R₄, R₅, R₆, R₇, R₈, R₉, and R₁₀ are each independently selected from the group consisting of H, alkyl, hydroxyl, oxyalkyl, alkyloxy, alkylthio, halo, aryl, and Y, wherein at least one of R₁, R₂, R₃, R₄, R₅, R₆, R₇, R₈, R₉, and R₁₀ is Y, and Y is selected from the group consisting of: 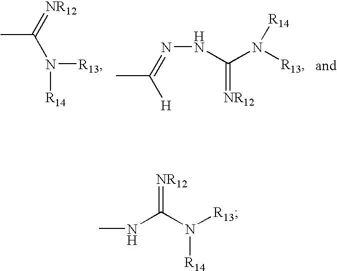
wherein:

R₁₂ is selected from the group consisting of H, hydroxyl, cycloalkyl, aryl, aralkyl, alkoxyl, hydroxycycloalkyl, alkoxycycloalkyl, hydroxyalkyl, aminoalkyl, acyloxy, and alkylaminoalkyl;

R₁₃ and R₁₄ are each independently selected from the group consisting of H, hydroxyl, alkyl, alkoxyalkyl, cycloalkyl, aryl, aralkyl, hydroxyalkyl, aminoalkyl, and alkylaminoalkyl;

or R₁₃ and R₁₄ together are:

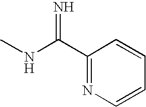

or R₁₂ and R₁₃ together represent a C₂ to C₁₀ alkyl, hydroxyalkyl, or alkylene;

or R₁₂ and R₁₃ together are: 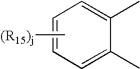
wherein:

j is an integer from 1 to 3, and R₁₅ is H or Y, as set forth above.

It is accordingly an object of the presently disclosed subject matter to provide compounds that are useful in the treatment of microbial infections. It is another object to provide pharmaceutical formulations for use in the treatment of microbial infections. It is still another object to provide methods for treating microbial infections.

Certain objects having been stated hereinabove, which are addressed in whole or in part by the presently disclosed subject matter, other aspects and objects will become evident as the description proceeds when taken in connection with the accompanying examples as best described herein below.

### DETAILED DESCRIPTION

The presently disclosed subject matter will be now be described more fully hereinafter with reference to the accompanying Examples, in which preferred embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety.

Throughout the specification and claims, a given chemical formula or name shall encompass all optical and stereoisomers as well as racemic mixtures where such isomers and mixtures exist.

### I. DEFINITIONS

As used herein the term “alkyl” refers to C₁₋₂₀ inclusive, linear (i.e., “straight-chain”), branched, or cyclic, saturated or unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl). “Higher alkyl” refers to an alkyl group having about 10 to about 20 carbon atoms. In certain embodiments, “alkyl” refers, in particular, to C₁₋₈ straight-chain alkyls. In other embodiments, alkyl refers, in particular, to C₁₋₈ branched-chain alkyls.

Alkyl groups can optionally be substituted with one or more alkyl group substituents, which can be the same or different. The term “alkyl group substituent” includes but is not limited to alkyl, halo, arylamino, acyl, hydroxy, aryloxy, alkoxyl, alkylthio, arylthio, aralkyloxyl, aralkylthio, carboxyl, alkoxycarbonyl, oxo and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

The term “aryl” is used herein to refer to an aromatic substituent which may be a single aromatic ring, or multiple aromatic rings that are fused together, linked covalently, or linked to a common group such as a methylene or ethylene moiety. The common linking group may also be a carbonyl as in benzophenone or oxygen as in diphenylether or nitrogen in diphenylamine. The term “aryl” specifically encompasses heterocyclic aromatic compounds. The aromatic ring(s) may comprise phenyl, naphthyl, biphenyl, diphenylether, diphenylamine and benzophenone, among others. In particular embodiments, the term “aryl” means a cyclic aromatic comprising about 5 to about 10 carbon atoms, including 5 and 6-membered hydrocarbon and heterocyclic aromatic rings.

The aryl group can be optionally substituted with one or more aryl group substituents which can be the same or different, where “aryl group substituent” includes alkyl, aryl, aralkyl, hydroxy, alkoxyl, aryloxy, aralkoxyl, carboxy, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene and —NR′R″, where R′ and R″ can be each independently hydrogen, alkyl, aryl and aralkyl.

Specific examples of aryl groups include but are not limited to cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole and the like.

Thus, as used herein, the terms “substituted alkyl” and “substituted aryl” include alkyl and aryl groups, as defined herein, in which one or more atoms or functional groups of the aryl or alkyl group are replaced with another atom or functional group, including for example, halogen, aryl, alkyl, alkoxyl, hydroxy, nitro, amino, alkylamino, dialkylamino, sulfate, and mercapto.

As used herein, the term “acyl” refers to an organic acid group wherein the —OH of the carboxyl group has been replaced with another substituent (i.e., as represented by RCO—, wherein R is an alkyl or an aryl group as defined herein). As such, the term “acyl” specifically includes arylacyl groups. Specific examples of acyl groups include acetyl and benzoyl.

“Cyclic” and “cycloalkyl” refer to a non-aromatic mono- or multicyclic ring system of about 4 to about 10 carbon atoms. The cycloalkyl group can be optionally partially unsaturated. The cycloalkyl group can be also optionally substituted with an alkyl group substituent as defined herein, oxo and/or alkylene. There can be optionally inserted along the cyclic alkyl chain one or more oxygen, sulfur or substituted or unsubstituted nitrogen atoms, wherein the nitrogen substituent is hydrogen, lower alkyl, or aryl, thus providing a heterocyclic group. Representative monocyclic cycloalkyl rings include cyclopentyl, cyclohexyl and cycloheptyl. Multicyclic cycloalkyl rings include adamantyl, octahydronaphthyl, decalin, camphor, camphane, and noradamantyl.

“Alkoxyl” or “Alkyloxyl” refer to an alkyl-O— group wherein alkyl is as previously described. The terms “alkoxyl” or “alkyloxyl” as used herein can refer to C₁₋₂₀ inclusive, linear, branched, or cyclic, saturated or unsaturated oxo-hydrocarbon chains, including, for example, methoxy, ethoxy, propoxy, isopropoxy, butoxy, t-butoxy, and pentoxy.

“Alkylthio” refers to an alkyl-S— group wherein alkyl is as previously described. The term “alkylthio” can refer to C₁₋₂₀ inclusive, linear, branched, or cyclic; saturated or unsaturated sulfur-hydrocarbon chains.

“Aryloxyl” refers to an aryl-O— group wherein the aryl group is as previously described. The term “aryloxyl” as used herein can refer to phenyloxyl or hexyloxyl, and alkyl, halo, or alkoxyl substituted phenyloxyl or hexyloxyl.

“Aralkyl” refers to an aryl-alkyl- group wherein aryl and alkyl are as previously described. Exemplary aralkyl groups include benzyl, phenylethyl and naphthylmethyl.

“Alkyloxyalkyl” refers to an alkyl-O— group wherein the alkyl group is as previously described.

“Aralkyloxyl” refers to an aralkyl-O— group wherein the aralkyl group is as previously described. An exemplary aralkyloxy group is benzyloxy.

“Aminoalkyl” refers to linear or branched amino-substituted alkyl, wherein the term “amino” refers to the group NR′R″, wherein R′ and R″ are independently selected from H or alkyl as defined above.

“Dialkylamino” refers to an —NRR′ group wherein each of R and R′ is independently an alkyl group as previously described. Exemplary alkylamino groups include ethylmethylamino, dimethylamino and diethylamino.

“Alkoxycarbonyl” refers to an alkyl-O—CO— group. Exemplary alkoxycarbonyl groups include methoxycarbonyl, ethoxycarbonyl, butyloxycarbonyl and t-butyloxycarbonyl.

“Aryloxycarbonyl” refers to an aryl-O—CO— group. Exemplary aryloxycarbonyl groups include phenoxy- and naphthoxy-carbonyl.

“Aralkoxycarbonyl” refers to an aralkyl-O—CO— group. An exemplary aralkoxycarbonyl group is benzyloxycarbonyl.

“Carbamoyl” refers to an H₂N—CO— group.

“Alkylcarbamoyl” refers to a R′RN—CO— group wherein one of R and R′ is hydrogen and the other of R and R′ is alkyl as previously described.

“Dialkylcarbamoyl” refers to R′RN—CO— group wherein each of R and R′ is independently alkyl as previously described.

“Acyloxyl” refers to an acyl-O— group wherein acyl is as previously described.

“Acylamino” refers to an acyl-NH— group wherein acyl is as previously described.

“Aroylamino” refers to an aroyl-NH— group wherein aroyl is as previously described.

“Alkylene” refers to a straight or branched bivalent aliphatic hydrocarbon group having from 1 to about 20 carbon atoms. The alkylene group can be straight, branched or cyclic. The alkylene group can be also optionally unsaturated and/or substituted with one or more “alkyl group substituents.” There can be optionally inserted along the alkylene group one or more oxygen, sulphur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)ₙ—N(R)—(CH₂)ₘ—, wherein each of m and n is independently an integer from 0 to about 20 and R is hydrogen or lower alkyl; methylenedioxy (—O—CH₂—O—); and ethylenedioxy (—O—(CH₂)₂—O—). An alkylene group can have about 2 to about 3 carbon atoms and can further have 6-20 carbons.

The terms “halo”, “halide”, or “halogen” as used herein refer to fluoro, chloro, bromo, and iodo groups.

The term “hydroxyl” as used herein refers to the —OH group.

The term “hydroxyalkyl” as used herein refers to a linear or branched hydroxy-substituted alkyl, i.e., —CH₂OH, —(CH₂)₂OH, etc., wherein alkyl is as previously described.

The term “oxy” as used herein refers to the substitution of an oxygen atom in a hydrocarbon chain.

The term “oxyalkyl” as used herein refers to oxygen-substituted alkyl, i.e., —OCH₃, wherein alkyl is as previously described.

When the term “independently selected” is used, the substituents being referred (i.e., R groups, such as groups R₁, and R₂, or groups X and Y), can be identical or different. For example, (e.g., R₂ and R₃ may both be substituted alkyls, or R₂ may be hydrogen and R₃ may be a substituted aryl, etc.).

A named “R”, “X,” “Y,” “A,” or “B” group will generally have the structure that is recognized in the art as corresponding to a group having that name, unless specified otherwise herein. For the purposes of illustration, certain representative “R,” “X,” “Y” groups as set forth above are defined below. These definitions are intended to supplement and illustrate, not preclude, the definitions known to those of skill in the art.

II. Novel Compounds

A. Compounds of Formula I

Described herein are compounds of Formula (I): 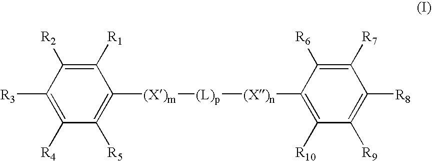
wherein:

X′ and X″ are each independently selected from the group consisting of alkyl, alkylene, oxygen, oxy, oxyalkyl, alkyloxy, alkyloxyalkyl, and 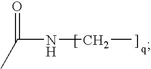
m, n, p, and q are each independently an integer from 0 to 10;

L is selected from the group consisting of hydroxyalkyl, 1,2-oxazole, 1,3-oxazole, phenyl, naphthyl, pyrimidine, alkyl-substituted pyrimidine and

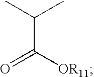

wherein R₁₁ is H or alkyl;

R₁, R₂, R₃, R₄, R₅, R₆, R₇, R₈, R₉, and R₁₀ are each independently selected from the group consisting of H, alkyl, hydroxyl, oxyalkyl, alkyloxy, halo, aryl, and Y, wherein at least one of R₁, R₂, R₃, R₄, R₅, R₆, R₇, R₈, R₉, and R₁₀ is Y, and Y is selected from the group consisting of:

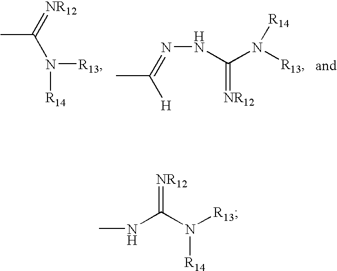

wherein:

R₁₂ is selected from the group consisting of H, hydroxyl, cycloalkyl, aryl, aralkyl, alkoxy, hydroxycycloalkyl, alkoxycycloalkyl, hydroxyalkyl, aminoalkyl, acyloxy, and alkylaminoalkyl;

R₁₃ and R₁₄ are each independently selected from the group consisting of H, hydroxyl, alkyl, alkoxyalkyl, cycloalkyl, aryl, aralkyl, hydroxyalkyl, aminoalkyl, and alkylaminoalkyl;

or R₁₂ and R₁₃ together represent a C₂ to C₁₀ alkyl, hydroxyalkyl, or alkylene;

or R₁₂ and R₁₃ together are:

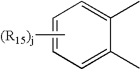

wherein:

j is an integer from 1 to 3, and R₁₅ is H or Y, as set forth above.

Particular embodiments of compounds of Formula I are illustrated by, but not limited to, those compounds described in Table 1.

TABLE 1
Amidine Compounds of Formula I.*

| | |
|---|---|
| | (I) |
| 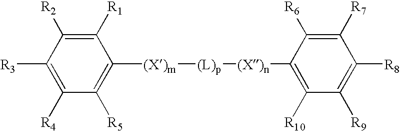 | |

| Cpd | m | n | p | L | X′ | X″ | R₂ | R₃ | R₄ | R₇ | R₈ |
|---|---|---|---|---|---|---|---|---|---|---|---|
| 1 | 1 | 1 | 1 | alkyl | 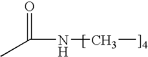 | 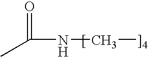 | H | Am | H | H | Am |
| 2 | 1 | 1 | 1 | hydroxyalkyl | oxyalkyl | oxyalkyl | H | Am | H | H | Am |
| 3 | 1 | 1 | 8 | methylene | oxygen | oxygen | H | Am | H | H | H |
| 4 | 0 | 0 | 1 | 1,2-oxazole | — | — | H | Am | H | Am | H |
| 5 | 0 | 0 | 1 | 1,2-oxazole | — | — | Am | H | H | H | Am |
| 6 | 0 | 1 | 1 | 1,3-oxazole | — | alkyl | H | Am | H | H | Am |
| 7 | 1 | 1 | 1 | phenyl | oxyalkyl | oxyalkyl | H | Im | H | H | Im |
| 8 | 1 | 1 | 1 | phenyl | oxyalkyl | oxyalkyl | H | Im | H | H | Im |
| 9 | 1 | 1 | 1 | phenyl | oxygen | oxygen | H | Am | H | H | Am |
| 10 | 1 | 1 | 1 | naphthyl | oxyalkyl | oxyalkyl | H | H | Isopropyl- | Isopropyl- | H |
| | | | | | | | | | Am | Am | |
| 11 | 1 | 1 | 1 | naphthyl | oxyalkyl | oxyalkyl | H | H | Isopropyl- | Isopropyl- | H |
| | | | | | | | | | Am | Am | |
| 12 | 1 | 1 | 1 | naphthyl | oxyalkyl | oxyalkyl | H | Isopropyl- | H | H | Isopropyl- |
| | | | | | | | | Am | | | Am |
| 13 | 1 | 1 | 1 | naphthyl | oxyalkyl | oxyalkyl | H | Isopropyl- | H | H | Isopropyl- |
| | | | | | | | | Am | | | Am |
| 14 | 1 | 1 | 1 | naphthyl | oxyalkyl | oxyalkyl | H | Isopropyl- | H | H | Isopropyl- |
| | | | | | | | | Am | | | Am |
| 15 | 1 | 1 | 1 | naphthyl | oxyalkyl | oxyalkyl | H | H | Am | Am | H |
| 16 | 1 | 1 | 1 | 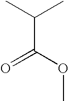 | oxyalkyl | oxyalkyl | H | amidoxime | H | H | amidoxime |
| 17 | 1 | 1 | 1 | alkyl | oxyalkyl | oxyalkyl | H | H | alkyl- | H | Isopropyl- |
| | | | | | | | | | benzamidol | | Am |
| 18 | 1 | 1 | 1 | alkyl | oxyalkyl | oxyalkyl | H | aryl-Am | H | H | aryl-Am |
| 19 | 1 | 0 | 0 | — | oxyalkyl | — | H | Am | H | H | H |
| 20 | 1 | 0 | 0 | — | oxyalkyl | — | H | Am | H | H | alkyl |
| 21 | 1 | 0 | 0 | — | oxyalkyl | — | H | H | Am | H | H |
| 22 | 0 | 0 | 1 | alkyl- | — | — | H | Am | H | H | Am |
| | | | | pyrimidine | | | | | | | |

*Unless otherwise noted each R group of Formula (I) is hydrogen.

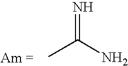

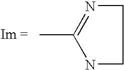

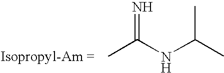

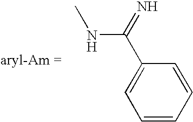

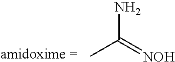

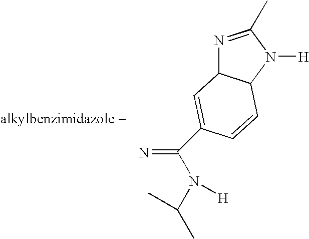

B. Compounds of Formula II

Also described herein are compounds of Formula (II): 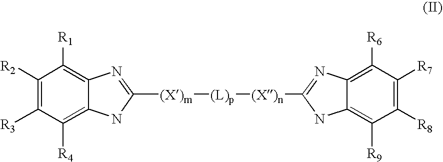
wherein:

m is an integer from 1 to 5;

n is an integer from 0 to 5;

p is an integer from 0 to 5;

X′ and X″ are each independently phenyl or thiophene;

L is selected from the group consisting of C₁₋₁₀ straight chain alkyl, C₁₋₁₀ branched chain alkyl, cycloalkyl, phenyl; naphthyl, and alkyl-substituted phenyl;

R₁, R₂, R₃, R₄, R₅, R₆, R₇, R₈, and R₉ are each independently selected from the group consisting of H, alkyl, hydroxyl, alkyloxy, oxyalkyl, halo, aryl, and Y, wherein at least one of R₁, R₂, R₃, R₄, R₅, R₆, R₇, R₈, and R₉ is Y, and Y is selected from the group consisting of:

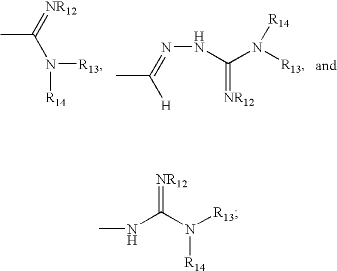

wherein:

R₁₂ is selected from the group consisting of H, hydroxyl, cycloalkyl, aryl, aralkyl, alkoxyl, hydroxycycloalkyl, alkoxycycloalkyl, hydroxyalkyl, aminoalkyl, acyloxyl, and alkylaminoalkyl;

R₁₃ and R₁₄ are each independently selected from the group consisting of H, hydroxyl, alkyl, alkoxyalkyl, cycloalkyl, aryl, aralkyl, hydroxyalkyl, aminoalkyl, and alkylaminoalkyl;

or R₁₂ and R₁₃ together represent a C₂ to C₁₀ alkyl, hydroxyalkyl, or alkylene;

or R₁₂ and R₁₃ together are:

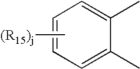

wherein:

j is an integer from 1 to 3, and R₁₅ is H or Y, as set forth above.

Particular embodiments of compounds of Formula II are illustrated by, but limited to, those compounds described in Table 2.

TABLE 2
Compounds of Formula II.*

| | |
|---|---|
| | (II) |
| 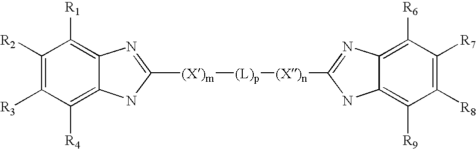 | |

| Cmpd | m | n | p | L | X′ | X″ | R₂ | R₃ | R₇ | R₈ |
|---|---|---|---|---|---|---|---|---|---|---|
| 23 | 0 | 0 | 1 | naphthyl | — | — | H | Am | H | Am |
| 24 | 1 | 1 | 2 | alkyl | phenyl | phenyl | H | Am | H | Am |
| 25 | 1 | 1 | 0 | — | phenyl | phenyl | H | Am | H | Am |
| 26 | 1 | 1 | 1 | alkyl | phenyl | phenyl | H | Am | H | Am |
| 27 | 1 | 1 | 1 | cyclopropane | phenyl | phenyl | H | Am | H | Am |
| 28 | 1 | 1 | 1 | alkyl | thiophene | phenyl | H | Am | H | Am |
| 29 | 0 | 0 | 1 | alkyl-phenyl | — | — | Am | H | Am | H |
| 30 | 1 | 1 | 1 | phenyl | alkyl | alkyl | Am | H | Am | H |
| 31 | 0 | 0 | 1 | phenyl | — | — | H | Am | H | Am |
| 32 | 1 | 1 | 2 | alkyl | phenyl | phenyl | alkyl- | H | alkyl- | H |
| | | | | | | | Am | | Am | |

*Unless otherwise noted each R group of Formula (II) is hydrogen.

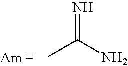

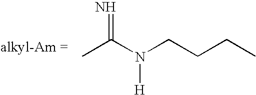

C. Compounds of Formula III
Also described herein are compounds of Formula (III): 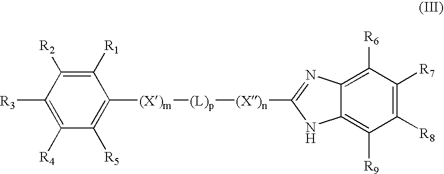
wherein:

L is phenyl, pyridine, or hydroxy-phenyl;

m and n are each independently an integer from 0 to 5;

X′ and X″ are each independently selected from the group consisting of C₁₋₁₀ straight chain alkyl, C₁₋₁₀ branched chain alkyl, and cycloalkyl;

R¹, R₂, R₃, R⁴, R₅, R₆, R₇, R₈, and R⁹ are each independently selected from the group consisting of H, alkyl, hydroxyl, alkyloxy, oxyalkyl, halo, aryl, and Y, wherein at least one of R₁, R₂, R₃, R₄, R₅, R₆, R₇, R₈, and R₉ is Y, and Y is selected from the group consisting of:

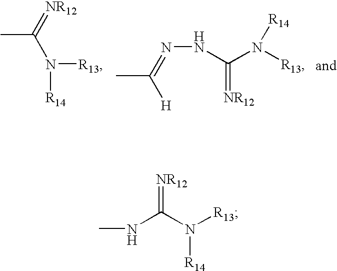

wherein:

R₁₂ is selected from the group consisting of H, hydroxyl, cycloalkyl, aryl, aralkyl, alkoxyl, hydroxycycloalkyl, alkoxycycloalkyl, hydroxyalkyl, aminoalkyl, acyloxyl, and alkylaminoalkyl;

R₁₃ and R₁₄ are each independently selected from the group consisting of H, hydroxyl, alkyl, alkoxyalkyl, cycloalkyl, aryl, aralkyl, hydroxyalkyl, aminoalkyl, and alkylaminoalkyl;

or R₁₂ and R₁₃ together represent a C₂ to C₁₀ alkyl, hydroxyalkyl, or alkylene;

or R₁₂ and R₁₃ together are:

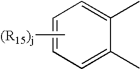

wherein:

j is an integer from 1 to 3, and R₁₅ is H or Y, as set forth above.

Particular embodiments of compounds of Formula III are illustrated by, but not limited to, those compounds described in Table 3.

TABLE 3
Amidine Compounds of Formula III.*

| | |
|---|---|
| | (III) |
| 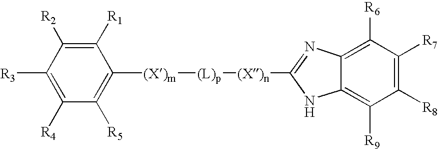 | |

| Compound | m | n | p | L | X′ | X″ | R₃ | R₈ |
|---|---|---|---|---|---|---|---|---|
| 33 | 1 | 0 | 1 | phenyl | alkyl | — | alkoxyl | Am |
| 34 | 1 | 0 | 1 | phenyl | alkyl | — | alkyl | Am |
| 35 | 1 | 0 | 1 | phesnyl | alkyl | — | halo | Am |
| 36 | 0 | 0 | 1 | pyridine | — | — | Am | Am |
| 37 | 0 | 0 | 1 | hydroxy- | — | — | Am | Am |
| | | | | phenyl | | | | |

*Unless otherwise noted each R group of Formula (I) is hydrogen.

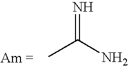

D. Compounds of Formula IV

Also described herein are compounds of Formula (IV):

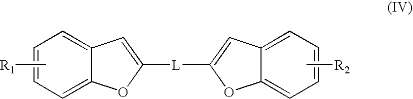

wherein L is selected from the group consisting of C₂₋₁₀ straight chain alkyl, C₁₋₁₀ branched chain alkyl, and cycloalkyl;

R₁ and R₂ are selected from the group consisting of:

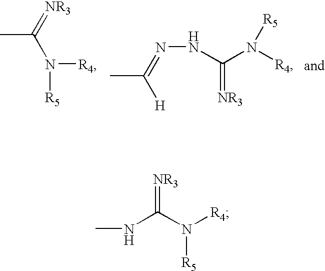

wherein R₃ is selected from the group consisting of H, hydroxyl, cycloalkyl, aryl, aralkyl, alkoxyl, hydroxycycloalkyl, alkoxycycloalkyl, hydroxyalkyl, aminoalkyl, acyloxyl, and alkylaminoalkyl;

R₄ and R₅ are each independently selected from the group consisting of H, hydroxyl, alkyl, alkoxyalkyl, cycloalkyl, aryl, aralkyl, hydroxyalkyl, aminoalkyl, and alkylaminoalkyl;

or R₃ and R₄ together represent a C₂ to C₁₀ alkyl, hydroxyalkyl, or alkylene;

or R₄and R₅together are:

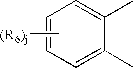

wherein:

j is a number from 1 to 3, and R₆ is selected from the group consisting of H and the groups from which R₁ and R₂ may be selected.

In particular embodiments of compounds of Formula IV, L is alkyl and R₁ and R₂ are each: 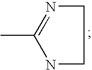
for example, compound 38, which has the following structure:

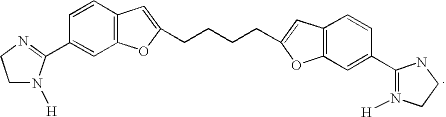

In other embodiments of compounds of Formula IV, L is alkyl and R₁ and R₂ are: 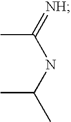
for example, compound 39, which has the following structure:

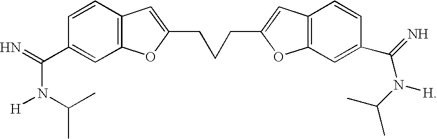

E. Compounds of Formula V

Also described herein are compounds of Formula (V):

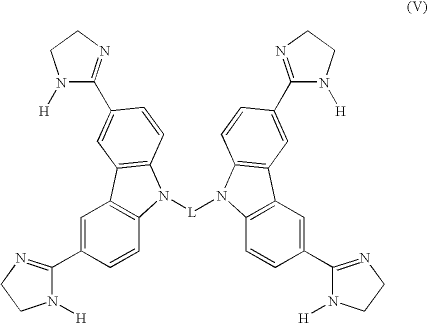

In particular embodiments of compounds of Formula V, L is alkyl, for example, compound 40, which has the following structure: 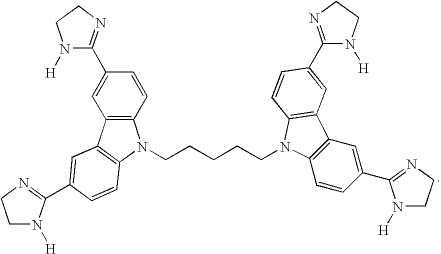
F. Compounds of Formula VI

Also described herein are compounds of Formula VI: 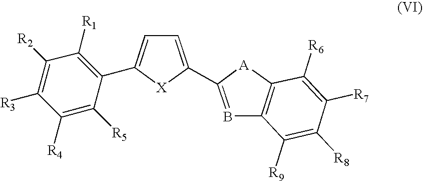
wherein:

X is oxygen;

A and B are each either nitrogen or oxygen;

R₁, R₂, R₃, R₄, R₅, R₆, R₇, R₈, and R₉ are each independently selected from the group consisting of H, alkyl, hydroxyl, alkyloxy, oxyalkyl, halo, aryl, and Y, wherein at least one of R₁, R₂, R₃, R₄, R₅, R₆, R₇, R₈, and R₉ is Y, and

Y is selected from the group consisting of:

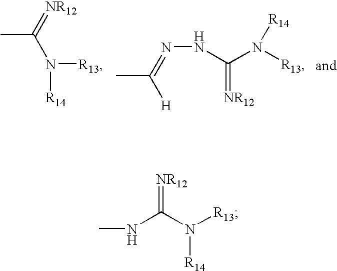

wherein:

R₁₂ is selected from the group consisting of H, hydroxyl, cycloalkyl, aryl, aralkyl, alkoxyl, hydroxycycloalkyl, alkoxycycloalkyl, hydroxyalkyl, aminoalkyl, acyloxyl, and alkylaminoalkyl;

R₁₃ and R₁₄ are each independently selected from the group consisting of H, hydroxyl, alkyl, alkoxyalkyl, cycloalkyl, aryl, aralkyl, hydroxyalkyl, aminoalkyl, and alkylaminoalkyl;

or R₁₂ and R₁₃ together represent a C₂ to C₁₀ alkyl, hydroxyalkyl, or alkylene;

or R₁₂ and R₁₃together are:

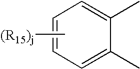

wherein:

j is an integer from 1 to 3, and R₁₅ is H or Y, as set forth above.

In particular embodiments of compounds of Formula VI, X and A are each oxygen, B is nitrogen, and R₃ and R₈ are each: 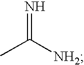
for example, compound 41, which has the following structure: 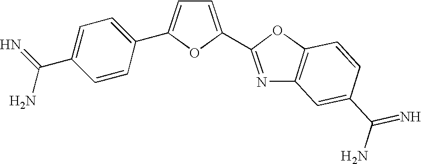
G. Compounds of Formula (VII)

Also described herein are compounds of Formula (VII): 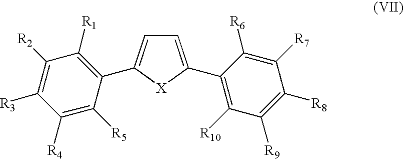
wherein:

X is oxygen; and

R₁, R₂, R₃, R₄, R₅, R₆, R₇, R₈, R₉, and R₁₀ are each independently selected from the group consisting of H, alkyl, hydroxyl, oxyalkyl, alkyloxy, alkylthio, halo, aryl, and Y, wherein at least one of R₁, R₂, R₃, R₄, R₅, R₆, R₇, R₈, R₉, and R₁₀ is Y, and Y is selected from the group consisting of: 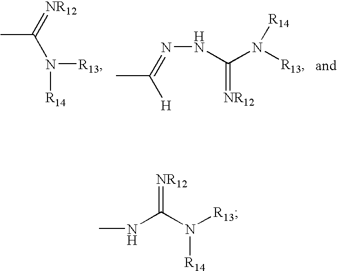
wherein:

R₁₂ is selected from the group consisting of H, hydroxyl, cycloalkyl, aryl, aralkyl, alkoxy, hydroxycycloalkyl, alkoxycycloalkyl, hydroxyalkyl, aminoalkyl, acyloxy, and alkylaminoalkyl;

R₁₃ and R₁₄ are each independently selected from the group consisting of H, hydroxyl, alkyl, alkoxyalkyl, cycloalkyl, aryl, aralkyl, hydroxyalkyl, aminoalkyl, and alkylaminoalkyl;

or R₁₃ and R₁₄ together are:

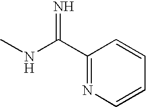

or R₁₂ and R₁₃ together represent a C₂ to C₁₀ alkyl, hydroxyalkyl, or alkylene;

or R₁₂ and R₁₃ together are: 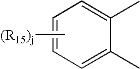
wherein:

j is an integer from 1 to 3, and R₁₅ is H or Y, as set forth above.

In particular embodiments of compounds of Formula VII, X is oxygen, R₂ and R₇ are alkylthio, and R₃ and R₈ are each: 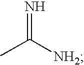
for example, compound 42, which has the following structure:

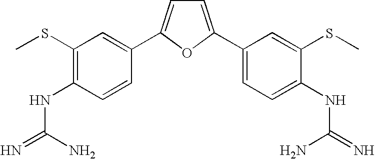

In another embodiment of compounds of Formula VII, X is oxygen, R₁ and R₆ are hydroxy, and R₃ and R₈ are each: 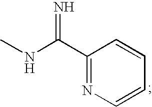
for example, compound 43, which has the following structure:

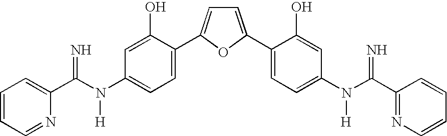

H. Prodrugs

In representative embodiments, compounds disclosed herein are prodrugs. A prodrug means a compound that, upon administration to a recipient, is capable of providing (directly or indirectly) a compound of the presently disclosed subject matter or an inhibitorily active metabolite or residue thereof. Prodrugs can increase the bioavailability of the compounds of the presently disclosed subject matter when such compounds are administered to a subject (e.g., by allowing an orally administered compound to be more readily absorbed into the blood) or can enhance delivery of the parent compound to a biological compartment (e.g., the brain or lymphatic system) relative to a metabolite species. By way of example, Compound 16 described herein is a prodrug.

I. Pharmaceutically Acceptable Salts

Additionally, the active compounds can be administered as pharmaceutically acceptable salts. Such salts include the gluconate, lactate, acetate, tartarate, citrate, phosphate, borate, nitrate, sulfate, and hydrochloride salts. The salts of the compounds described herein can be prepared, in general, by reacting two equivalents of the base compound with the desired acid, in solution. After the reaction is complete, the salts are crystallized from solution by the addition of an appropriate amount of solvent in which the salt is insoluble. In a particular embodiment, the pharmaceutically acceptable salt is an acetate salt.

III. Pharmaceutical Formulations

The compounds of Formulae I-VII, the pharmaceutically acceptable salts thereof, prodrugs corresponding to compounds of Formulae I-VII, and the pharmaceutically acceptable salts thereof, are all referred to herein as “active compounds.” Pharmaceutical formulations comprising the aforementioned active compounds are also provided herein. These pharmaceutical formulations comprise active compounds as described herein, in a pharmaceutically acceptable carrier. Pharmaceutical formulations may be prepared for oral, intravenous, or aerosol administration as discussed in greater detail below. Also, the presently disclosed subject matter provides such active compounds that have been lyophilized and that can be reconstituted to form pharmaceutically acceptable formulations for administration, as by intravenous or intramuscular injection.

The therapeutically effective dosage of any specific active compound, the use of which is in the scope of embodiments described herein, will vary somewhat from compound to compound, and patient to patient, and will depend upon the condition of the patient and the route of delivery. As a general proposition, a dosage from about 0.1 to about 50 mg/kg will have therapeutic efficacy, with all weights being calculated based upon the weight of the active compound, including the cases where a salt is employed. Toxicity concerns at the higher level may restrict intravenous dosages to a lower level such as up to about 10 mg/kg, with all weights being calculated based upon the weight of the active base, including the cases where a salt is employed. A dosage from about 10 mg/kg to about 50 mg/kg may be employed for oral administration. Typically, a dosage from about 0.5 mg/kg to 5 mg/kg may be employed for intramuscular injection. Preferred dosages are 1 μmol/kg to 50 μmol/kg, and more preferably 22 μmol/kg and 33 μmol/kg of the compound for intravenous or oral administration. The duration of the treatment is usually once per day for a period of two to three weeks or until the condition is essentially controlled. Lower doses given less frequently can be used prophylactically to prevent or reduce the incidence of recurrence of the infection.

In accordance with the present methods, pharmaceutically active compounds as described herein can be administered orally as a solid or as a liquid, or can be administered intramuscularly or intravenously as a solution, suspension, or emulsion. Alternatively, the compounds or salts can also be administered by inhalation, intravenously or intramuscularly as a liposomal suspension. When administered through inhalation the active compound or salt should be in the form of a plurality of solid particles or droplets having a particle size from about 0.5 to about 5 microns, and preferably from about 1 to about 2 microns.

Pharmaceutical formulations suitable for intravenous or intramuscular injection are further embodiments provided herein. The pharmaceutical formulations comprise a compound of Formulae I-VII described herein, a prodrug as described herein, or a pharmaceutically acceptable salt thereof, in any pharmaceutically acceptable carrier. If a solution is desired, water is the carrier of choice with respect to water-soluble compounds or salts. With respect to the water-soluble compounds or salts, an organic vehicle, such as glycerol, propylene glycol, polyethylene glycol, or mixtures thereof, can be suitable. In the latter instance, the organic vehicle can contain a substantial amount of water. The solution in either instance can then be sterilized in a suitable manner known to those in the art, and typically by filtration through a 0.22-micron filter. Subsequent to sterilization, the solution can be dispensed into appropriate receptacles, such as depyrogenated glass vials. Of course, the dispensing is preferably done by an aseptic method. Sterilized closures can then be placed on the vials and, if desired, the vial contents may be lyophilized.

In addition to compounds of Formulae I-VII or their salts or prodrugs, the pharmaceutical formulations can contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the formulations can contain anti-microbial preservatives. Useful anti-microbial preservatives include methylparaben, propylparaben, and benzyl alcohol. The anti-microbial preservative is typically employed when the formulation is placed in a vial designed for multi-dose use. The pharmaceutical formulations described herein can be lyophilized using techniques well known in the art.

In yet another aspect of the subject matter described herein, there is provided an injectable, stable, sterile formulation comprising a compound of any one of Formulae I-VII, or a salt thereof, in a unit dosage form in a sealed container. The compound or salt is provided in the form of a lyophilizate, which is capable of being reconstituted with a suitable pharmaceutically acceptable carrier to form a liquid formulation suitable for injection thereof into a subject. The unit dosage form typically comprises from about 10 mg to about 10 grams of the compound salt. When the compound or salt is substantially water-insoluble, a sufficient amount of emulsifying agent, which is physiologically acceptable, can be employed in sufficient quantity to emulsify the compound or salt in an aqueous carrier. One such useful emulsifying agent is phosphatidyl choline.

Other pharmaceutical formulations can be prepared from the water-insoluble compounds disclosed herein, or salts thereof, such as aqueous base emulsions. In such an instance, the formulation will contain a sufficient amount of pharmaceutically acceptable emulsifying agent to emulsify the desired amount of the compound or salt thereof. Particularly useful emulsifying agents include phosphatidyl cholines, and lecithin.

Additional embodiments provided herein include liposomal formulations of the active compounds disclosed herein. The technology for forming liposomal suspensions is well known in the art. When the compound is an aqueous-soluble salt, using conventional liposome technology, the same can be incorporated into lipid vesicles. In such an instance, due to the water solubility of the active compound, the active compound will be substantially entrained within the hydrophilic center or core of the liposomes. The lipid layer employed can be of any conventional composition and can either contain cholesterol or can be cholesterol-free. When the active compound of interest is water-insoluble, again employing conventional liposome formation technology, the salt can be substantially entrained within the hydrophobic lipid bilayer that forms the structure of the liposome. In either instance, the liposomes that are produced can be reduced in size, as through the use of standard sonication and homogenization techniques.

The liposomal formulations containing the active compounds disclosed herein can be lyophilized to produce a lyophilizate, which can be reconstituted with a pharmaceutically acceptable carrier, such as water, to regenerate a liposomal suspension.

Pharmaceutical formulations are also provided which are suitable for administration as an aerosol, by inhalation. These formulations comprise a solution or suspension of a desired compound described herein or a salt thereof, or a plurality of solid particles of the compound or salt. The desired formulation can be placed in a small chamber and nebulized. Nebulization can be accomplished by compressed air or by ultrasonic energy to form a plurality of liquid droplets or solid particles comprising the compounds or salts. The liquid droplets or solid particles should have a particle size in the range of about 0.5 to about 10 microns, more preferably from about 0.5 to about 5 microns. The solid particles can be obtained by processing the solid compound or a salt thereof, in any appropriate manner known in the art, such as by micronization. Most preferably, the size of the solid particles or droplets will be from about 1 to about 2 microns. In this respect, commercial nebulizers are available to achieve this purpose. The compounds can be administered via an aerosol suspension of respirable particles in a manner set forth in U.S. Pat. No. 5,628,984, the disclosure of which is incorporated herein by reference in its entirety.

When the pharmaceutical formulation suitable for administration as an aerosol is in the form of a liquid, the formulation will comprise a water-soluble active compound in a carrier that comprises water. A surfactant can be present, which lowers the surface tension of the formulation sufficiently to result in the formation of droplets within the desired size range when subjected to nebulization.

As indicated, both water-soluble and water-insoluble active compounds are provided. As used in the present specification, the term “water-soluble” is meant to define any composition that is soluble in water in an amount of about 50 mg/mL, or greater. Also, as used in the present specification, the term “water-insoluble” is meant to define any composition that has solubility in water of less than about 20 mg/mL. For certain applications, water-soluble compounds or salts can be desirable whereas for other applications water-insoluble compounds or salts likewise can be desirable.

IV. Methods of Treating Microbial Infections

Subjects with microbial infections can be treated by methods described herein. These infections can be caused by a variety of microbes, including fungi, algae, protozoa, bacteria, and viruses. Exemplary microbial infections that can be treated by the method of the presently disclosed subject matter include, but are not limited to, infections caused by Trypanosoma species (e.g., Trypanosoma brucei rhodesiense), Pnemocytsis carnii, Giardia lamblia, Cryptosporidium parvum, Cryptococcus neoformans, Candida albicans, Candida tropicalis, Salmonella typhimurium, Plasmodium falciparum, Leishmania donovani, and Leishmania mexicana amazonensis. The methods of the presently disclosed subject matter are useful for treating these conditions in that they inhibit the onset, growth, or spread of the condition, cause regression of the condition, cure the condition, or otherwise improve the general well-being of a subject afflicted with, or at risk of contracting the condition.

Methods of treating microbial infections comprise administering to a subject in need of treatment an active compound as described herein. These active compounds, as set forth above, include compounds of Formulae I-VII, their corresponding prodrugs, and pharmaceutically acceptable salts of the compounds and prodrugs. With regard to the presently described method embodiments, compounds of Formulae I-VII are defined as having the structures of Formulae I-VII as defined above.

The subject treated in the presently disclosed subject matter in its many embodiments is desirably a human subject, although it is to be understood the methods described herein are effective with respect to all vertebrate species, which are intended to be included in the term “subject”. The methods described herein are particularly useful in the treatment and/or prevention of infectious diseases in warm-blooded vertebrates. Thus, the methods may be used as treatment for mammals and birds.

More particularly, provided is the treatment of mammals such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, i.e., poultry, such as turkeys, chickens, ducks, geese, guinea fowl, and the like, as they are also of economical importance to humans. Thus, embodiments of the methods described herein include the treatment of livestock, including, but not limited to, domesticated swine (pigs and hogs), ruminants, horses, poultry, and the like.

Background methods of treating microbial infections are described in U.S. Pat. Nos. 6,503,940; 6,486,200; 6,326,395; 6,294,565; 6,172,104; 6,156,779; 6,127,554; 6,046,226; 6,017,941; 6,008,247; 5,972,969; 5,939,440; 5,935,982; 5,817,687; 5,817,686; 5,792,782; 5,668,167; 5,668,166; 5,643,935; 5,639,755; 5,602,172; 5,578,631; and 5,428,051; each of which are incorporated herein by reference in their entirety.

### EXAMPLES

The following Examples have been included to illustrate modes of the presently disclosed subject matter. Certain aspects of the following Examples are described in terms of techniques and procedures found or contemplated to work well in the practice of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Methods and Materials For Examples 1-9

Melting points were recorded using a Thomas-Hoover (Uni-Melt) capillary melting point apparatus and are uncorrected. TLC analysis was carried out on silica gel 60 F₂₅₄ precoated aluminum sheets and detected under UV light. ¹H and ¹³C NMR spectra were recorded employing a Varian GX400 or Varian Unity Plus 300 spectrometer, and chemical shifts (δ) are in ppm relative to TMS as internal standard. Mass spectra were recorded on a VG analytical 70-SE spectrometer for pure components. Elemental analyses were obtained from Atlantic Microlab Inc. (Norcross, Ga., United States of America) and are within ±0.4 of the theoretical values. All chemicals and solvents were purchased from Aldrich Chemical Co. or Fisher Scientific or Frontier or Lancaster.

The synthesis of amidine compounds of the presently disclosed subject matter is described in U.S. Pat. Nos. 5,428,051, 4,963,589, 5,202,320, 5,935,982, 5,521,189, 5,686,456, 5,627,184, 5,622,955, 5,606,058, 5,668,167, 5,667,975, 6,025,398, 6,214,883, 5,817,687, 5,792,782, 5,939,440, 6,017,941, 5,972,969, 6,046,226, 6,294,565 (B1), 6,156,779, 6,326,395, 6,008,247, 6,127,554, 6,172,104, 4,940,723, 5,206,236, 5,843,980, 4,933,347, 5,668,166, 5,817,686, 5,723,495, 4,619,942, 5,792,782, 5,639,755, 5,643,935, 5,602,172, 5,594,138, and 5,578,631, each of which are incorporated herein by reference in their entirety. The compounds disclosed herein can also be synthesized according to art-recognized techniques.

### Example 1

### 2.6-Diformyl-naphthalene

To a stirred solution of 3.5 g (0.02 mole) of 2,6-dicyanonaphthalene in 75 mL CH₂Cl₂ under N₂was added DIBAL (4.26 g, 30 mL, 1 M solution in cyclohexane) in 10 min., after 15 min. stirring, it was heated at 45° C. for 45 min. The cooled reaction mixture (ice-bath) was decomposed with 2N H₂SO₄ (50 mL) while stirring continued for 1 h, CH₂Cl₂ layer was separated, washed with water, NaHCO₃, water and dried over Na₂SO₄ and filtered and conc. in vac. triturated with hexane and filtered and dried to yield 2.66 g (72.3%), pale crystalline solid, m.p. 173-4° C.; ¹H-NMR (DMSO-d₆): 10.18 (s, 1H), 10.17 (s, 1H), 8.57 (s, 2H), 8.23 (d, 2H, J=8.4 Hz), 7.96 (d, 2H, J=8.4 Hz); ¹³CNMR (DMSO-d6): 192.5, 135.6, 134.9, 133.0, 130.2, 123.4; MS: m/e 184 (M⁺).

### 2,6-Bis{2-[(4-amidino)benzimidazolyl]}-naphthalenetetrahydrochloride (Compound 23, DB-464)

The above dialdehyde (0.184 g, 0.001 mole), 4-amidino1,2-phenylenediamine hydrochloride hemihydrate (0.39 g, 0.002 mole) and 0.216 g (0.002 mole) 1,4-benzoquinone in ethanol was refluxed for 12 h and after standard work-up was converted to its hydrochloride salt, 0.43 g (70%); m.p. >300° C.; ¹H-NMR (DMSO-d₆): 8.87 (s, 2H), 8.42 (d, 2H, J=8.4 Hz), 8.28 (d, 2H, J=8.4 Hz), 8.23 (s, 2H), 7.86 (d, 2H, J=8.4 Hz), 7.74 (d, 2H, J=8.4 Hz); FAB MS: m/e 445 (M⁺+1); ¹³CNMR (DMSO-d₆): 166.7, 145.2, 141.2, 138.4, 134.3, 130.6, 127.9, 127.1, 125.5, 123.6, 122.9, 116.5, 115.8; Anal. calc. for C₂₆H₂₀N₈.4HCl.1.5H₂O. C, 52.89; H, 4.40: N, 18.98. Found: C, 52.51; H, 4.53; N, 18.86.

### Example 2

### 4,4′-Bis{2-[-6(2-imidazolino]benzimidazolyl}-1,2-diphenylethane tetrahydrochloride (Compound 24, DB-496)

A mixture of 4,4′-diformyl-1,2-diphenylethane (0.238 g, 0.0001 mole), 4-amidino-1,2-phenylenediamine hydrochloride hemihydrate (0.39, 0.002 mole) and 1,4-benzoquinone (0.216, 0.002 mole) in 50 mL ethanol was refluxed under nitrogen for 12 h. After removing solvent residue diluted with water and stirred for 5 h, filtered, washed with water and dried. It was dissolved in hot methanol and filtered, acidified with methanolic-HCl (4 mL) and stirred, concentrated in vac, diluted with ether and dark solid filtered and dried in vac at 60° C. for 24 h, 0.42 g (64%). m.p. >300° C. dec. ¹HNMR (DMSO-d_{6/}D₂O): 8.20 (s, 2H), 8.09 (d, 4H, J=8 Hz), 7.87 (d, 2H, J=8.4 Hz), 7.78 (d, 2H, J=8.4 Hz), 7.49 (d, 2H, J-8 Hz), 3.06 (s, 4H). ¹³CNMR (DMSO-d₆): 166.1, 153.2, 147.2, 138.3, 135.1, 130.1, 128.2, 124.5, 123.9, 123.0, 115.6, 115.3, 36.64, FAB MS: m/e 499 (M⁺+1). Anal. calcd. for C₃₀H₂₆N₈.4HCl.H₂O (662.44). C, 54.39; H, 4.86; N, 16.91. Found: C, 54.42; H, 4.87; N, 16.93.

### Example 3

### 4.4′-Diformyl-1.1′-biphenyl

To a stirred solution of 2.04 g (0.01 mole) of 4,4′-dicyanobiphenyl in 75 mL CH₂Cl₂ under N₂ was added DIBAL (4.36 g, 30 mL, 1 M solution in cyclohexane) in 10 min., after 15 min. stirring, it was heated at 45° C. for 45 min. The cooled reaction mixture (ice-bath) was decomposed with aq. 2NH₂SO₄ (50 mL) while stirring continued for 1hr, CH₂Cl₂ layer was separated, washed with water, NaHCO₃, water and dried over Na₂SO₄ anhd., filtered and conc. in vac., triturated with hexane and filtered and dried to yield 1.4 g (67%) pale crystalline solid, m.p. 165-8° C.; ¹H-NMR (DMSO-d₆): 10.07 (s, 2H), 7.99 (d, 4H, J=8.4 Hz), 7.92 (d, 4H, J=8.4 Hz); ¹³CNMR (DMSO-d6): 192.4, 144.2, 135.7, 129.9, 127.7; MS: m/e 210 (M⁺).

### 4.4′-Bis{2-[(4-Amidino)benzimidazolyl]}biphenyl tetrahydrochloride (Compound 25, DB 507)

The above dialdehyde (0.21 g, 0.001 mole), 0.39 g (0.002 mole), 4-amidino-1,2-phenylenediamine hydrochloride hemihydrate and (0.216 g, 0.002 mole) 1,4-benzoquinone in ethanol was refluxed for 12 hr and after standard work-up was converted to its hydrochloride salt, 0.43 g (66%); m.p. >300° C. dec.; ¹H-NMR (DMSO-d₆): 8.35 (d, 4H, J=7.6 Hz), 8.21 (s, 2H), 8.02 (d, 4H, J=7.6 Hz), 7.85 (d, 4H, J=8.4 Hz), 7.50 (d, 4H, J=8.4 Hz); ¹³CNMR (DMSO-d6): 166.0, 153.2, 141.4, 137.5, 128.4, 127.8, 126.9, 123.4, 122.6, 116.2, 115.1; FAB MS: m/e 483 (M⁺+1); Analysis calculated for C₂₉H₂₂N₈.4HCl.1.5H₂O: C, 53.41; H, 4.46: N, 17.09. Found: C, 52.97; H, 4.61; N,7.17.

### Example 4

### 2-(4-Bromophenyl)-3-[2-(5-bromothienyl)acrylonitrile]

A few drops of 5N. NaOH (aq) was added to a boiling solution of 5-bromo-thiophene-2-aldehyde (8.55 g, 0.05 m) and 4-bromophenylacetonitrile (9.8, 0.05 mole) in 25 mL CH₃OH, an exothermic reaction resulted to a solid mass, cooled diluted with water filtered, dissolved in CHCl₃, dried over anhydr. Na₂SO₄ filtered and con., triturated with ether:hexane and filtered, bright yellow/green 170-72° C.; ¹HNMR (DMSO-d₆): 8.15 (s, 1H), 7.64 (A₂B₂q, 4H, J=8.4 Hz), 7.55 (d, 1H, J=3.6 Hz), 7.35 (d, 1H, J=3.6 Hz); ¹³CNMR (DMSO-d₆): 138.8, 135.05, 135.02, 132.1, 131.8, 131.1, 127.2, 122.1, 117.5, 117.2, 105.7; MS m/e 369 (M⁺), for C₁₃H₇Br₂NS.

### 2-(4-Bromophenyl)-3-[2-(5-bromothienyl)]-propionitrile

A suspension of the above acrylonitrile analog. (14.76 g, 0.04 mole) in 100 mL CH₃OH and 50 mL pyridine was reduced by adding (4.5 g, 0.12 mole) sodium borohydride, heated under reflux for 30 min., excess solvent distilled, cooled and acidified while stirring with conc. HCl, solid filtered, washed with water, redissolved in CHCl₃, dried over Na₂SO₄ filtered with ether:hexane to yield a white solid (12.6 g, 85%), m.p. 64-6° C.; ¹HNMR (DMSO-d₆), 8.58 (d, 2H, J=8.4 Hz), 8.35 (d, 2H, J=8.4 Hz), 8.02 (d, 1H, J=4 Hz), 7.48 (d, ₁H, J=4 Hz), 5.56 (t, 1H, J=6.8 Hz), 4.50-4.30 (m, 2H); ¹³CNMR (DMSO-d₆): 146.8, 134.2, 131.6, 129.8, 129.6, 127.8, 121.2, 119.8, 109.5, 36.9, 34.0; MS m/e 371 (M⁺), for C₁₃H₉Br₂NS.

### 2-(4-Bromophenyl)-3-[2-(5-bromophenyl)]propionic acid

A mixture of the above nitrile 11.13 g (0.03 mole) in 150 mL 20% aq. NaOH and 15 mL ethanol was heated at reflux for 7 h, diluted with water, cooled, acidified with HCl to pH=3, the precipitated acid was filtered, washed with water, dried and crystallized from benzene:hexane as white solid 9.3 g (79%), m.p. 110-111° C.; ¹HNMR (DMSO-d₆): 7.49 (d, 2H, J=8.4), 7.27 (d, 2H, J=8.4 Hz), 6.93 (d, 1H, J=3.6 Hz), 6.63 (d, 1H, J=3.6 Hz), 3.84 (t, 1H, J=7.6 Hz), 3.44 (dd, 1H, J=7.6, J=23.2 Hz), 3.16 (dd, 1H, J=7.6, J=23.2 Hz); ¹³CNMR (DMSO-d₆), 172.7, 143.1, 137.6, 130.9, 129.8, 129.5, 126.4, 120.1, 108.2, 51.6, 32.7; MS: m/e 390 (M⁺), for C₁₃H₁₀Br₂O₂S

### 1-(4-Cyanophenyl)-2-[2-(5-cyanothienyl)]ethane

A mixture of the above acid (11.7 g, 0.03 mole) and Cu(I)CN (8.01 g, 0.09 mole) in 35 mL dry N-methyl-2-pyrolidone was heated for 1.5 h, cooled, stirred for 2 h with 200 mL 10% NaCN, filtered washed with water, the solid was dissolved in 10 mL acetone, passed through a neutral alumina column and eluted with CHCl₃ followed by CHCl₃:Acetone to yield 5.2 g (73%) pale yellow brown solid 116-8° C.; ¹HNMR (DMSO-d₆), 7.72 (d, 1H, J=3.6 Hz), 7.71 (d, 2H, J=8 Hz), 7.43 (d, 2H, J=8 Hz), 7.0 (d, 1H, J=3.6 Hz), 3.23 (t, 2H, J=7.6 Hz), 3.05 (t, 2H, J=7.6 Hz); ¹³CNMR (DMSO-d₆): 152.4, 145.8, 138.5, 131.9, 129.3, 126.2, 118.5, 114.0, 108.9, 105.6, 36.1, 29.8; MS m/e 238 (M⁺); Analysis C₁₄H₁₀N₂S (238.3), C, 70.56; H, 4.23; N, 11.75. Found C, 70.83; H, 4.12; N, 11.63

### 1-(4-Formylphenyl)-2-[2-(5-formylthienyl)]ethane

To a stirred solution of the above dinitrile (2.38 g, 0.01 mole) in 75 ml CH₂Cl₂ under N₂ was added DIBAL (4.36 g, 30 mL, 1 M solution in cyclohexane) over 10 min., after 15 min. stirring, it was heated at 45° C. for 45 min. The cooled reaction mixture (ice-bath) was decomposed with 2N H₂SO₄ (50 mL) while stirring continued for 1 hr. The CH₂Cl₂ layer was separated, washed with water, NaHCO₃, water and dried over Na₂SO₄ (and.), filtered and conc. in vac. triturated with hexane and filtered and dried to yield 1.6 g (65%), yellow solid, m.p. 106-8° C.; ¹HNMR (CDCl₃): 9.97 (s, 1H), 9.80 (s, 1H), 7.79 (d, 2H, J=8 Hz), 7.57 (d, 1H, J=4 Hz), 7.32 (d, 2H, J=8 Hz), 6.83 (d, 1H, J=4 Hz), 3.23 (t, 2H, J=7.6 Hz), 3.10 (t, 2H, J=7.6 Hz); ¹³CNMR (CDCl₃), 191.5, 182.3, 154.8, 147.1, 142.3, 136.5, 135.2, 130.0, 129.1, 126.4, 37.4, 31.9; MS m/e 244 (M⁺); Analysis C₁₄H₁₂O₂S (244.31), C, 68.78; H, 4.94. Found: C, 68.41; H, 4.89.

### 1-{4-[2-[(5-Amidino)benzimidazolyl]phenyl}-2-{5-[2-(5-amidino)benzimidazoly]thienyl}ethane trihydrochloride (Compound 28, DB 598)

The above dialdehyde (0.24 g, 0.001 mole), 0.39 g (0.002 mole) 4-amidino,1,2-phenylenediamine hydrochloride hemihydrate and 0.216 g (0.002 mole) 1,4-benzoquinone in ethanol was refluxed for 12 h and after standard work-up was converted to its hydrochloride salt, 0.39 g (58%), m.p. >310° C. dec.; ¹HNMR (DMSO-d₆/D₂O), 8.09 (brs, 1H), 8.02 (d, 2H, J=8.4 Hz), 7.98 (brs, 1H), 7.80 (d, 1H, J=8.4 hz), 7.70-7.63 (m, 3H), 7.61 (dd, 1H, J=1.2 Hz, J=8.4 Hz), 7.48 (d, 1H, J=8.4 Hz), 6.98 (d, 1H, J=4 Hz); FAB MS: m/e 505 (M⁺+1); Anal. calcd. for C₂₈H₂₄N₈S.3HCl.H₂O. C, 53.21; H, 4.62; N, 17.72. Found: C, 53.58; H, 4.79; N, 17.52.

### Example 5

### 2,5-Bis(3-ethoxy-4-guanidinophenyl)furan dihydrochloride (Compound 44, DB779)

2-Nitro-5-bromophenetole (64% yield; mp, 78 to 79° C. [ethanol-water]) was produced by the reaction of 3,4-dinitrobromobenzene with sodium ethoxide in ethanol. Coupling of the bromo compound with 2,5-bis(tributylstannyl)furan gave, after recrystallization from N,N-dimethylformamide-methanol, 2,5-bis(3-3-ethoxy-4-nitrophenyl)furan as a yellow-orange fluffy solid (75% yield; mp, 192 to 194° C., ¹H NMR (DMSO-d₆): 1.38 (t, 6H), 4.34 (q, 4H), 7.51 (s, 2H), 7.59 (dd, J=8.4, 1.8, 2H), 7.69 (d, J=1.8 Hz, 2H), 7.97 (d, J=8.7, 2H). Analysis calculated for C₂₀H₁₈N₂O₇ (398.36): C, 60.30; H, 4.55; N, 7.03. Found: C, 60.34; H, 4.58; N, 6.93.

Hydrogenation with Pd on C gave, after crystallization from methanol-water, 2,5-bis(4-amino-3-ethyoxyphenyl)furan as a light green and tan solid (85% yield). ¹H NMR (DMSO-d₆): 1.36 (t, 6H), 4.07 (q, 4H), 4.85 (br s, 4H), 6.63 to 6.68 (m, 4H), 7.10 (m, 4H). From the diamine, the title bis-guanidine was prepared as a light green hygroscopic solid (76% yield for a two-step procedure). ¹H NMR (DMSO-d₆): 1.38 (q, 6H), 4.21 (d, 2H), 7.27 (dd, J=8.1, 2.1, 2H), 7.42 (br s, 8H), 7.44 to 7.49 (m, 4H), 9.40 (br s, 2NH). Mass spectrum (electrospray): m/e 423.3 (60% yield: M⁺−2HCl). Analysis calculated for C₂₂H₂₆N₆O₃.2HCl.0.5H₂O (504.41): C, 52.38; H, 5.79; N, 16.67. Found: C, 52.25; H, 5.81; N, 16.52. See, e.g., M. D. Givens, C. C. Dykstra, K. V. Brock, D. A. Stringfellow, A. Kumar, C. E. Stephens, H. Goker, D. W. Boykin In Vitro Inhibition of Replication of Bovine Viral Diarrhea Virus by Aromatic Cationic Molecules, Antimicrobial Agents and Chemotherapy, 47, 2223-2230 (2003).

### Compound 6

### 6-(4-Cyanophenyl)pyridine-2-carbaldehyde (1)

To a solution of 6-bromopyridine-2-carbaldehyde (3.85 g, 20.7 mmol) and Pd(PPh₃)₄ (0.70 g, 0.6 mmol) in 40 mL of toluene under a nitrogen atmosphere was added 20 mL of 2 M aqueous Na₂CO₃ and 3.30 g (22.7 mmol) of 4-cyanobenzeneboronic acid in 10 mL of methanol. The mixture was vigorously stirred at 80° C. overnight. The mixture was cooled and extracted with dichloromethane. The organic layer was dried and concentrated to dryness under reduced pressure to give 2.60 g (60%) of product, mp 158-159° C. ¹H-NMR (DMSO-d₆) δ 10.17 (s, 1H), 8.24 (d, 2H, J=8.0), 8.00 (m, 3H), 7.82 (d, 2H, J=8.0); ¹³C-NMR (DMSO-d₆) δ 188.9, 151.3, 148.7, 137.8, 133.9, 128.4, 123.2, 120.3, 116.5, 114.2, 108.9; MS (EI) calcd. mass for C₁₃H₈N₂O: 208.2; observed mass 208.1. Anal. calcd. for C₁₃H₈N₂O: C, 74.99; H, 3.87; N, 13.45. Found: C, 75.12; H, 3.89; N, 13.35.

### 2-(5-Cyanobenzimidazol-2-yl)-6-(4-cyanophenyl)pyridine (2)

A solution of 6-(4-cyanophenyl)pyridine-2-carbaldehyde (1), (3.0 g, 14.4 mmol), 3,4-diaminobenzonitrile (1.89 g, 14.4 mmol) and benzoquinone (1.55 g, 14.4 mmol) in 240 mL of ethanol was heated at reflux under a nitrogen atmosphere overnight. After cooling the solid was collected by filtration. The solid was heated at reflux for 2 h in a mixture ether/ethanol. Cooling and filtration afforded 2.51 g (56%) of a beige solid, mp 311-312° C. ¹H-NMR (DMSO-d₆), δ 8.63 (d, 2H, J=8.0), 8.37 (d, 1H, J=7.0), 8.29 (d, 1H, J=7.0), 8.17 (dd, 1H, J=8.0 and 7.0), 8.07 (d, 3H, J=8.0), 7.83 (d, 1H, J=7.0), 7.69 (d, 1H, J=8.0); ¹³C-NMR (DMSO-d₆), δ 154.0, 153.9, 153.3, 147.5, 141.7, 139.0, 132.6, 132.6, 127.6, 126.0, 122.3, 121.6, 119.7, 112.0, 104.5; HRMS (EI) calcd. mass for C₂₀H₁₁N₅: 321.335; observed mass 321.101.

### 2-(5-Hydroxyamidinobenzimidazol-2-yl)-6-(4-hydroxyamidinophenyl) pyridine(3)

To a solution of hydroxylamine hydrochloride (2.60 g, 37 mmol) in 20 mL of DMSO potassium t-butoxide (4.20 g, 37 mmol) was added in portions under nitrogen. After stirring the mixture for 30 min, 1.20 g (3.7 mmol) of 2-(5-cyanobenzimidazol-2-yl)-6-(4-cyanophenyl)pyridine (2) was added and the mixture was stirred at room temperature overnight. The mixture was poured into ice water and filtratered to yield the expected 2-(5-hydroxyamidinobenzimidazol-2-yl)-6-(4-hydroxyamidinophenyl)pyridine as a white solid (1.45 g, quantitative yield); mp >290° C. ¹H-NMR (DMSO-d₆), δ 9.79 (s, 1H), 9.60 (d, 1H), 8.44 (d, 2H, J=8.0), 8.28 (d, 1H, J=8.0), 8.16 (d, 1H, J=8.0), 8.08 (d, 1H, J=8.0), 8.04 (s, 1H), 7.88 (d, 2H, J=8.0), 7.68 (d, 1H, J=8.0), 7.60 (d, 1H, J=8.0), 5.96 (s, 2H), 5.86 (s, 2H); ¹³C-NMR (DMSO-d₆), δ 155.2, 150.39, 150.38, 148.07, 148.06, 138.4, 138.01, 138.00, 134.17, 134.15, 126.5, 125.5, 120.7, 120.1, 118.6, 111.4; MS (FAB), calcd. mass for C₂₀H₁₇N₇O₂ (M+H): 388.4; observed mass 388.1. Anal. calcd. for C₂₀H₁₇N₇O₂-0.6H₂O: C, 60.32; H, 4.61; N, 24.62. Found: C, 60.71; H, 4.65; N, 24.24.

### 2-(5-Acetoxyamidinobenzimidazol-2-yl)-6-(4-acetoxyamidino phenyl)pyridine (4)

The above amidoxime (3) (0.35 g, 0.9 mmol) was dissolved in glacial acetic acid (5 mL) and acetic anhydride (0.5 mL, 6.5 mmol) was added.⁶ The mixture was stirred for 2 h during which time the product precipitates. The product was filtratered and dried overnight in an oven. A white solid was obtained in 90% yield (0.38 g), mp 150-153 ° C. ¹H-NMR (DMSO-d₆), δ 8.51 (d, 2H, J=8.0), 8.33 (d, 1H, 8.0), 8.22 (d, 1H, J=8.0), 8.12 (t, 1H, J=8), 8.08(s, 1H), 7.93 (d, 2H, J=8.0), 7.74 (d, 1H, J=8.0), 7.67 (d, 1H, J=8.0), 6.95 (s, 2H), 6.87 (s, 2H), 2.17 (s, 3H), 2.16 (s, 3H), 1.91 (s, 2H); ¹³C-NMR (DMSO-d₆), δ 172.0, 171.9, 168.6, 168.53, 168.50, 157.12, 156.00, 154.9, 151.9, 147.9, 139.5, 138.8, 132.6, 127.1, 126.8, 121.4, 120.7, 21.0, 19.9, 19.8; MS (FAB), calcd. mass for C₂₄H₂₁N₇O₄ (M+H): 472.5; observed mass 472.2. Anal. calcd. for C₂₄H₂₁N₇O₄-0.65CH₃COOH-0.5H₂O: C, 58.60; H, 4.76; N 18.98. Found: C, 58.45; H, 4.70; N, 18.65.

### 2-(5-Amidinobenzimidazol-2-yl)-6-(4-amidinophenyl)pyridine acetate salt (Compound 36, DB 509)

A suspension of the preceding acetoxy compound (4) (0.3 g, 0.6 mmol) in acetic acid (20 mL) was hydrogenated over 10% palladium on carbon (0.20 g, 1.90 mmol) on a Parr apparatus at room temperature until the uptake of hydrogen ceased Filtration over a celite pad and evaporation of the solvent afforded the product in a 90% yield (0.30 g), mp >300° C. ¹H-NMR (DMSO-d₆), δ 8.66 (d, 2H, J=8.4), 8.38 (d, 1H, J=7.6), 8.31 (d, 1H, J=6.9), 8.18 (m, 2H, J=7.2), 8.00 (d, 2H, J=8.4), 7.85 (d, 1H, J=7.2), 7.70 (d, 1H, J=7.2), 1.81 (s, 9H); 13C-NMR (DMSO-d₆), δ 166.4, 165.3, 165.2, 154.3, 153.4, 147.9, 142.4, 139.1, 128.72, 128.67, 128.4, 127.4, 122.5, 122.2, 121.7, 121.6, 18.5; MS (FAB), calcd. mass for C₂₀H₁₇N₇ (M+H): 356.4; observed mass 356.1. Anal. calcd. for C₂₀H₁₇N₇-3CH₃CO₂H-1.5H₂O: C, 55.50; H, 5.73; N, 17.43. Found: C, 55.09; H, 5.70; N, 17.23.

### Example 7

### 5-Bromo-2-nitrothioanisole

A room-temperature solution of 4-bromo-1,2-dinitrobenzene (11.15 g, 45.1 mmol) in dry EtOH (100 mL) was prepared by heating, followed by quickly cooling in an ice/water bath. Sodium thiomethoxide (3.39 g, 48.4 mmol) was then added in one portion with stirring. The resulting brown/burgundy mixture was stirred at room-temperature for 1.5 h, and then brought to reflux. Once boiling, the heat was removed and the suspension was allowed to stir for 30 minutes. The resulting yellow/orange suspension was diluted with water (75 mL) and stored in the freezer for 1 h. The solid product was then collected and recrystallized from EtOH (500 mL, followed by concentration to 300 mL) to yield an orange solid (5.54 g, 50%). A second recrystallization from EtOH gave the pure product as orange micro-needles (5.00 g, 45%), mp 163-164.5° C. ¹H NMR (DMSO-d₆): 2.56 (s, 3H), 7.57 (dd, J=2.0, 8.8 Hz), 7.67 (d, J=1.9 Hz, NOE enhanced upon irradiation of the SMe signal at 2.56 ppm), 8.14 (d, J=8.8 Hz). IR (KBr, cm⁻¹): 3104, 3081, 2986, 2920, 1580, 1552, 1502, 1329, 1288, 1088, 856, 748, 671, 522. Anal. Calcd. for C₇H₆BrNO₂S (248.10): C, 33.89; H, 2.44; N, 5.65. Found: C, 34.11, H, 2.46; N, 5.62.

### 2.5-Bis(4-nitro-3-thiomethoxyphenyl)furan

This compound was prepared according to a general literature procedure (1) by the coupling of 2,5-bis(trin-butylstannyl)furan (3.20 g, 5 mmol) with 5-bromo-2-nitrothioanisole (2.49 g, 10 mmol) in dioxane (25 mL). Recrystallization of the collected precipitate from DMF/EtOH gave an orange/red solid (1.32 g, 66%), mp 278-283° C. ¹H NMR (DMSO-d₆): 2.67 (s, 6H), 7.61 (s, 2H), 7.83 (dd, J=8.6, 1.6 Hz, 2H), 7.88 (s, 2H), 8.30 (d, J=8.8 Hz, 2H). Anal. Calcd. for C₁₈H₁₄N₂O₅S₂ (402.43): C, 53.72; H, 3.51; N, 6.96. Found: C, 53.85; H, 3.68; N, 7.07.

### 2.5-Bis(4-amino-3-thiomethoxyphenyl)furan

A mixture of the above nitro compound (1.29 g, 3.2 mmol) and SnCl₂.2H₂O (5.80 g, 25.7 mmol) in dry EtOH (100 mL) and DMSO (20 mL) was heated under nitrogen for 20 h. The mixture was then basified with concentrated NaOH solution (chilling) and extracted with EtOAc. The extract was washed with water, then brine, and then dried (Na₂SO₄). To the filtered extract was added silica gel and the solvent was removed in vacuo. The product/silica gel was subjected to column chromatography (SiO₂) eluting with 20% EtOAc in hexanes. The homogeneous red fraction was concentrated to give a tan/red solid, which was triturated with hexanes and collected. Yield: 0.74 g (68%), mp 132-134. ¹H NMR (DMSO-d₆): 2.37 (s, 6H), 5.38 (s, 2NH), 6.67 (s, 2H), 6.75 (d, J=8.4 Hz, 2H), 7.39 (dd, J=2.0, 8.4 Hz, 2H), 7.55 (d, J=1.8 Hz, 2H).

### 2,5-Bis(4-guanidino-3-thiomethoxyphenyl)furan Dihydrochloride (Compound 42. DB815)

This compound was prepared from the above diamine (0.31 g, 0.9 mmol) using a standard, two-step procedure for synthesis of similar guanidines as outlined in the literature (1) (and above for DB762). The intermediate Di-Boc guanidine was obtained as a pale yellow solid (0.42 g, 56%) following column chromatography (SiO₂, 10% EtOAc in hexanes). ¹H NMR (CDCl₃): 1.54 (s, 36H), 2.44 (s, 6H), 6.67 (s, 2H), 7.62 (dd, J=1.6, 8.4 Hz, 2H), 7.76 (s, 2H), 8.35 (d, J=8.6 Hz, 2H). Treatment with dry HCl in EtOH/CH₂Cl₂ gave the title product as a tan solid in quantitative yield (0.25 g). ¹H NMR (DMSO-d₆): 2.58 (s, 6H), 7.29 (s, 2H), 7.31 (d, J=8.0 Hz, 2H), 7.41 (br s, 8NH), 7.67-7.71 (m, 4H), 9.57 (br s, 2NH). Anal. Calcd. for C₂₀H₂₂N₂OS₂.2HCl.0.75H₂O (512.98): C, 46.82; H, 5.01; N, 16.38. Found: C, 47.18; H, 5.09; N, 15.99.

### Example 8

### 5-[(4-Cyano-2-methyl)-phenyl]-5-(4-formylphenyl)-furan

A suspension of 4-amino-3-methylbenzonitrile (5 g, 0.038 mole) in 35 mL water and 5 mL conc. HCl was diazotized at 0° C. with a solution of (3.9 g, 0.056 mole) NaNO₂ in 10 mL water, allowed to stir at 0° C. for 30 min. The diazotized mixture was added slowly with stirring to a solution of 2-Furfuraldehyde (3.9 g, 0.042 mole) and CuCl₂.2H₂O (10 mole %) in 20 mL acetone and 30 mL water in 30 min., allowed to stir at .t. for 12 h precipitated brown solid was filtered and washed with water till free from blue color. It was dissolved in hot ethanol, treated with charcoal and filtered, triturated with ether and after standing yielded 0.43 g (54%) white crystalline solid, m.p. 206-8° C. ¹H-NMR (DMSO-d₆): 9.68 (s, 1H), 7.94 (d, 1H, J=8.1 Hz), 7385 (d, 1h, J=1.2), 7.78 (dd, 1H, J=1.2 Hz, J=7.1 Hz), 7.68 (d,1H, J=3.9 Hz), 7.26 (d, 1H, J=3.9 Hz), 2.56 (s, 3H); ¹³CNMR (DMSO-d6): 178.4, 155.7, 152.0, 136.7, 134.8, 132.2, 129.9, 128.2, 124.1, 118.3, 113.8, 111.4; MS: m/e 211 (M⁺).

### 2-{2-[5(6)-Cyano]benzimidazolyl}-5-[(4-cyano-2-methyl)-phenyl]-furan

A mixture of aldehyde (2.11 g, 0.01 mol), 4-cyano-1,2-phenylenediamine (1.33 g, 0.01 mol) and 1,4-benzoquinone (1.08 g, 0.01 mol) in 50 mL dry ethanol was heated under reflux under N₂ for 8 h. The reaction mixture was cooled and diluted with ether and filtered. The solid was collected and stirred with ethanol:ether (1:3) for 20 min. and the yellow brown solid was filtered, it was dissolved in hot methanol, filtered and concentrated in vac., diluted with ether and separated solid filtered, washed with ether and dried in vacuum at 70° C. for 12 h, 2.15 g (61%), m.p. 168-9° C. dec, ¹H-NMR (DMSO-d₆/D₂O): 8.10 (d, 1H, J=8 Hz), 8.07 (s, 1H), 7.78 (s, 1H), 7.77 (d, 1H, J=8 Hz), 7.73 (d, 1H, J=8 Hz), 7.57 (brd, 1H, J=8 Hz), 7.44 (d, 1H, J=3.6 Hz), 7.18 (d, 1H, J=3.6 Hz), 2.59 (s,3H). ¹³CNMR (DMSO-d6): 152.5, 146.5, 145.2, 142.0, 139.8, 135.8, 134.8, 132.8, 129.8, 127.4, 125.7, 120.4, 119.9, 118.6, 115.9, 114.2, 114.1, 110.3, 104.0, 21.3; MS: m/e 324 (M⁺1). Anal. calcd. for C₂₀H₁₂N₄O.1.5H₂O: C, 68.37; H, 4.30; N, 15.94. Found: C, 68.71; H, 4.16; N, 15.69

### 2-{2-[5(6)-Amidino]benzimidazolyl}-5-[(4-amidino-2-methyl)-phenyl]-furan trihydochloride (Compound 45, DB850)

The above dinitrile (2 g, 0.006 mole) in 75 mL ethanol was saturated with HCl gas at 0° C. and stirred at r.t. until TLC showed the disappearance of starting nitrile), diluted with ether and imidate ester hydrochloride was filtered, washed with ether and dried in vac at 30° C. for 5 h; 2.7 g (86%). 1.3 g (0.0019 mole) imidate ester hydrochloride was suspended in ethanol and saturated with ammonia at 0° C., stirred at r.t. for 24 h and after removing solvent diluted with ether:ethanol (6:1) and filtered. The yellow amidine was resuspended and treated with HCl gas to yield yellow amidine hydrochloride salt 0.57 g (57.5%), m.p. >290° C. dec. ¹HNMR (DMSO-d₆/D₂0), 8.15 (br, 1H), 8.12 (d, 1H, J=1.5), 7.98-7.60 (m, 3H), 7.67 (dd, 1H, J=1.5, J=7.5), 7.50 (d, 1H, J=3.6), 7.19 (d, 1H, J=3.6), 2.62 (s, 3H); ¹³CNMR (DMSO-d₆/D₂0), 166.4, 165.4, 153.6, 146.1, 144.6, 142.0, 139.5, 135.9, 133.7, 131.3, 127.8, 127.1, 126.2, 122.9, 122.1, 116.7, 115.5, 115.2, 114.5, 22.0; FAB MS: m/e 359 (M⁺+1); Anal. calcd for C₂₀H₁₈N₆O.3HCl.3.5H₂O; C, 45.25; H, 5.32; N, 15.38. Found: C, 44.94; H, 5.28; N, 15.37.

### Example 9

### 2,5-Bis(2-benzyloxy-4-nitrophenyl)furan

This compound was prepared according to a general literature procedure (1) by the coupling of 2,5-bis(tri-n-butylstannyl)furan (1.60 g, 2.5 mmol) with 3-benzyloxy-4-bromonitrobenzene (1.54 g, 5 mmol) in dioxane (10 mL). Recrystallization of the collected precipitate from DMF/EtOH gave an orange solid (0.98 g, 75%), mp 233-237° C. ¹H NMR (DMSO-d₆): 5.45 (s, 4H), 7.24 (s, 2H), 7.38-7.45 (m, 6H), 7.53 (d, J=7.3 Hz, 4H), 7.92 (dd, J=2.0, 8.6 Hz, 2H), 8.01 (d, J=2.2 Hz, 2H), 8.18 (d, J=8.6 Hz, 2H).

### 2,5-Bis(4-amino-2-hydroxyphenyl)furan

A suspension of the above nitro compound (0.96 g, 1.8 mmol) and Pd/C (10%) (0.10 g) in EtOAc (40 mL) and dry EtOH (10 mL) was hydrogenated at 50 psi until hydrogen uptake subsided (4 h). After the catalyst was removed by filtration over Celite, the solution was concentrated in vacuo to give a gummy orange solid. Trituration with ether gave a light brown/orange solid (0.52 g, quantitative), mp >150° C. dec. ¹H NMR (DMSO-d₆): 5.09 (s, 4H), 6.10-6.15 (m, 4H), 6.58 (s, 2H), 7.39 (d, J=8.2 Hz, 2H), 9.46 (s, 2OH).

### 2,5-Bis[2-hydroxy-4-(2-pyridylimino)amino]furan Dihydrochloride (Compound 43. DB750)

This compound was prepared according to a general literature procedure (1) by reaction of the above diamine (0.282 g, 1.0 mmol) with S-(2-naphthylmethyl)-2-pyridylthioimidate hydrobromide (0.756 g, 2.1 mmol). The usual workup was employed to give a yellow solid after trituration with ether. Recrystallization from EtOH/water gave the pure free-base as a yellow/olive solid (0.34 g, 69%), mp 163.5-165° C. The title salt was prepared by treating an EtOH solution of the free-base with dry HCl, followed by concentrating the solution in vacuo to near dryness to give a suspension. After diluting with ether, the red/orange solid was collected and dried in vacuo. ¹H NMR (DMSO-d₆): 7.02 (d, J=7.9 Hz, 2H), 7.15 (m, 4H), 7.83 (dd, J=4.6, 7.5 Hz, 2H), 8.03 (d, J=8.3 Hz, 2H), 8.20 (m, 2H), 8.43 (d, J=7.9 Hz, 2H), 8.88 (d, J=4.6 Hz, 2H), 9.30 (br s, NH), 10.04 (br s, NH), 10.94 (s, 2OH), 11.76 (br s, NH). Anal. Calcd. for C₂₈H₂₂N₆O₃.2HCl.1.5H₂O (590.46): C, 56.95; H, 4.61; N, 14.23; Cl, 12.01. Found: C, 57.02; H, 4.71; N, 13.93; Cl, 12.00.

### Example 10

Table 4 shows in vitro data for certain compounds of Formulae I-VI. In particular, Table 4 shows the effectiveness of certain compounds of Formulae I-VII against Trypanosoma brucei rhodesiense (T.b.r.) and Plasmodium falciparum (P.f.). Certain compounds were shown to be effective for treating T.b.r. in vivo. These compounds can thus be employed as treatments of second-stage human African trypanosomiasis.

TABLE 4
Effectiveness of Compounds of Formulae I-VII against Trypanosoma
brucei rhodesiense and Plasmodium falciparum.

| | IC50 (nM) vs. | In vivo vs. T.b.r. | |
|---|---|---|---|
| Compound No. | T.b.r. | cures | IC50 (nM) vs. P.f. |
| 6 | 21.7 | | 25.5 |
| 24 | 393 | | 19.6 |
| 26 | 262 | | 21 |
| 27 | 15 | | 9.3 |
| 44 | 40 | | 14.7 |
| 36 | 24 | 1/4 | 9.7 |
| 42 | 57 | | 66 |
| 41 | 11 | 4/4 | 32 |
| 37 | 17 | 4/4 | 131 |
| 45 | 9.4 | 2/4 | 147 |
| 43 | 32 | | 5.1 |

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A compound having the general formula: 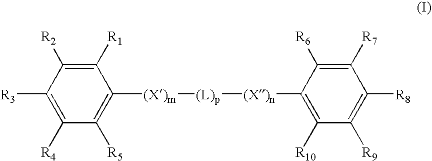
wherein:
X′ and X″ are each independently selected from the group consisting of alkyl, alkylene, oxygen, oxy, oxyalkyl, alkyloxy, alkyloxyalkyl, and 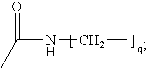
m, n, p, and q are each independently an integer from 0 to 10;
L is selected from the group consisting of hydroxyalkyl, 1,2-oxazole, 1,3-oxazole, phenyl, naphthyl, pyrimidine, alkyl-substituted pyrimidine and 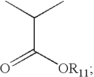
wherein R₁₁ is H or alkyl;
R₁, R₂, R₃ R₄, R₅ R₆, R₇, R₈, R₉, and R₁₀ are each independently selected from the group consisting of H, alkyl, hydroxyl, oxyalkyl, alkyloxy, halo, aryl, and Y, wherein at least one of R₁, R₂, R₃ , R₄, R₅, R₆, R₇, R₈, R₉, and R₁₀ is Y, and Y is selected from the group consisting of: 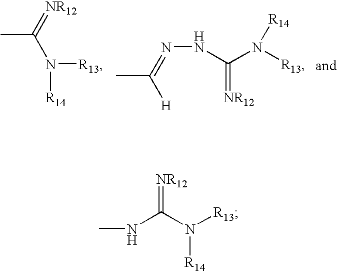
wherein:
R₁₂ is selected from the group consisting of H, hydroxyl, cycloalkyl, aryl, aralkyl, alkoxyl, hydroxycycloalkyl, alkoxycycloalkyl, hydroxyalkyl, aminoalkyl, acyloxy, and alkylaminoalkyl;
R₁₃ and R₁₄ are each independently selected from the group consisting of H, hydroxyl, alkyl, alkoxyalkyl, cycloalkyl, aryl, aralkyl, hydroxyalkyl, aminoalkyl, and alkylaminoalkyl;
or R₁₂ and R₁₃ together represent a C₂ to C₁₀ alkyl, hydroxyalkyl, or alkylene;
or R₁₂ and R₁₃ together are: 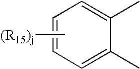
wherein:
j is an integer from 1 to 3, and R₁₅ is H or Y, as set forth above.

2. The compound according to claim 1, wherein:
p, m and n are each 1;
L is alkyl;
X′ and X″ are each 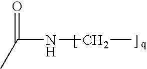
wherein q is an integer from 1 to 10; and
R₃ and R₈ are

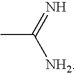

3. The compound according to claim 2, wherein the compound has the following structure:

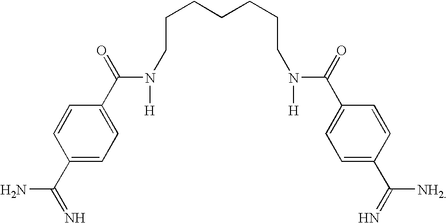

4. The compound according to claim 1, wherein:
m, n and p are each 1;
X′ and X″ are each oxyalkyl;
L is hydroxyalkyl;
nd R₃ and R₈ are

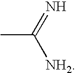

5. The compound according to claim 4, wherein the compound has the following structure:

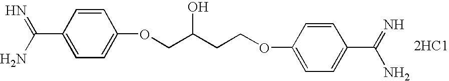

6. The compound according to claim 1, wherein:
m and n are 1;
p is 8;
X′ and X″ are each oxygen;
L is methylene; and
R₃ is: 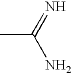
or a pharmaceutically acceptable salt thereof.

7. The compound according to claim 6, wherein the compound has the following structure:

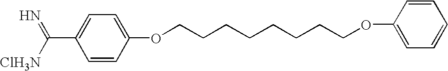

8. The compound according to claim 1, wherein:
m and n are 0;
p is 1;
L is 1,2-oxazole; and
R₃ and R₇ are

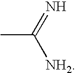

9. The compound according to claim 8, wherein the compound has the following structure:

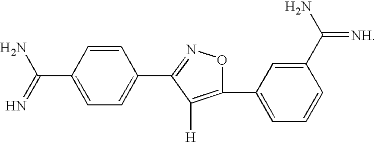

10. The compound according to claim 1, wherein:
m and n are 0;
p is 1;
L is 1,2-oxazole; and
R₂ and R₈ are

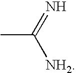

11. The compound according to claim 10, wherein the compound has the following structure:

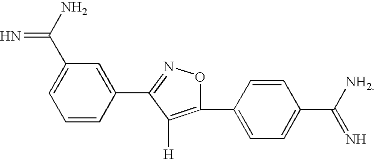

12. The compound according to claim 1, wherein:
m is 0;
n and p are each 1;
L is 1,3-oxazole;
X″ is alkyl; and
R₃ and R₈ are

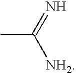

13. The compound according to claim 12, wherein the compound has the following structure:

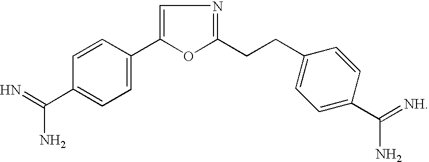

14. The compound according to claim 1, wherein:
m, n, and p are each 1;
L is phenyl;
X′ and X″ are each oxyalkyl; and
R₃ and R₈ are each

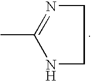

15. The compound according to claim 14, wherein the compound has the following structure:

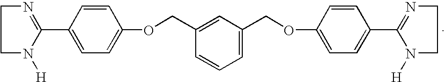

16. The compound according to claim 14, wherein the compound has the following structure:

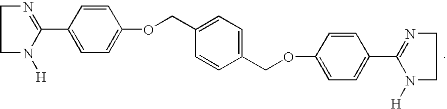

17. The compound according to claim 1, wherein:
m, n, and p are each 1;
L is phenyl;
X′ and X″ are each oxygen; and
R₃ and R₈ are each

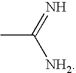

18. The compound according to claim 17, wherein the compound has the following structure:

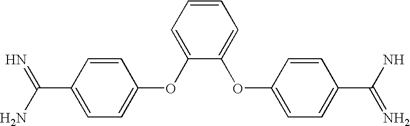

19. The compound according to claim 1, wherein:
m, n, and p are each 1;
L is naphthyl;
X′ and X″ are each oxyalkyl; and
R₄ and R₇ are each

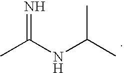

20. The compound according to claim 19, wherein the compound has the following structure:

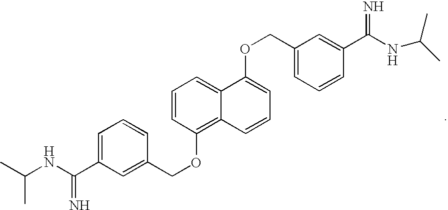

21. The compound according to claim 19, wherein the compound has the following structure:

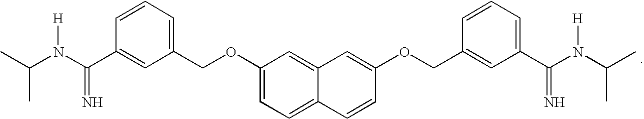

22. The compound according to claim 1, wherein:
m, n, and p are each 1;
L is naphthyl;
X′ and X″ are each oxyalkyl; and
R₃ and R₈ are each

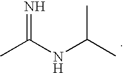

23. The compound according to claim 22, wherein the compound has the following structure:

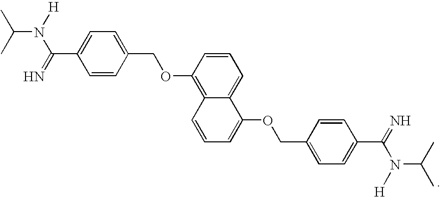

24. The compound according to claim 22, wherein the compound has the following structure:

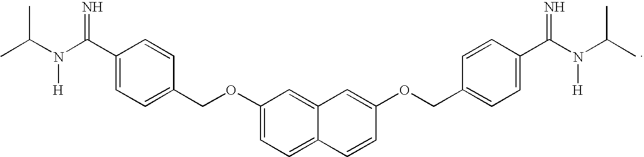

25. The compound according to claim 1, wherein:
m, n, and p are each 1;
L is naphthyl;
X′ and X″ are each oxyalkyl; and
R₃ and R₈ are each

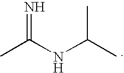

26. The compound according to claim 25, wherein the compound has the following structure:

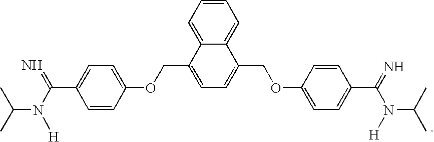

27. The compound according to claim 1, wherein:
m, n, and pare each 1;
L is naphthyl;
X′ and X″ are each oxyalkyl; and
R₄ and R₇ are each

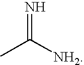

28. The compound according to claim 27, wherein the compound has the following structure:

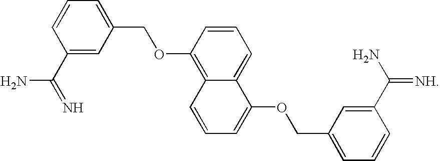

29. The compound according to claim 1, wherein:
m, n, and p are each 1;
L is 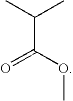
X′ and X″ are each oxyalkyl; and
R₃ and R₈ are each

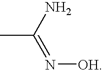

30. The compound according to claim 29, wherein the compound has the following structure:

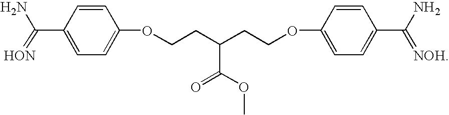

31. The compound according to claim 1, wherein:
p, m and n are each 1;
L is alkyl;
X′ and X″ are each oxyalkyl;
R₄ is alkyl-substituted benzimidazole; and
R₈is

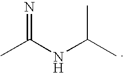

32. The compound according to claim 31, wherein the compound has the following structure:

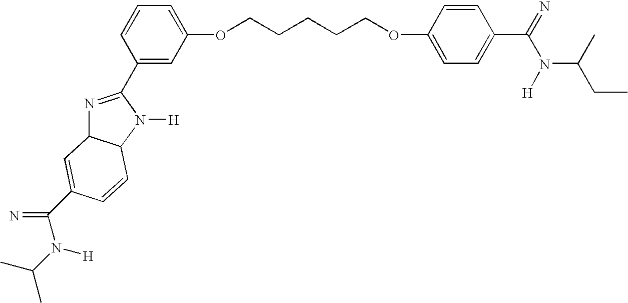

33. The compound according to claim 1, wherein:
p, m and n are each 1;
L is alkyl;
X′ and X″ are each oxyalkyl; and
R₃ and R₈ are each

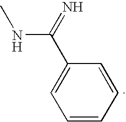

34. The compound according to claim 33, wherein the compound has the following structure:

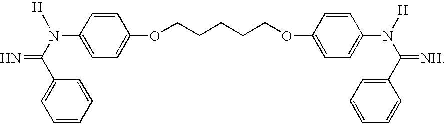

35. The compound according to claim 1, wherein:
p and n are each 0;
m is 1;
X′ is oxyalkyl; and
R₃ is 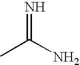
or a pharmaceutically acceptable salt thereof.

36. The compound according to claim 35, wherein the compound has the following structure:

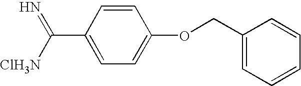

37. The compound according to claim 1, wherein:
n and p are each 0;
m is 1;
X′ is oxyalkyl;
R₈ is alkyl; and
R₃ is 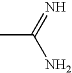
or a pharmaceutically acceptable salt thereof.

38. A compound according to claim 37, wherein the compound has the following structure:

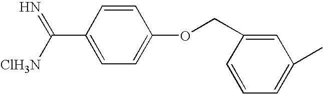

39. The compound according to claim 1, wherein:
n and p are each 0;
m is 1;
X′ is oxyalkyl;
R₈ is hydrogen; and
R₄ is

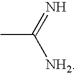

40. The compound according to claim 39, wherein the compound has the following structure:

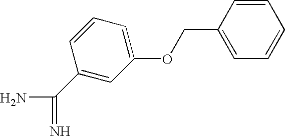

41. A compound according to claim 1, wherein:
n and m are each 0;
p is 1;
L is alkyl-substituted pyrimidine; and
R₃ and R₈ are each

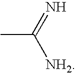

42. The compound according to claim 41, wherein the compound has the following structure:

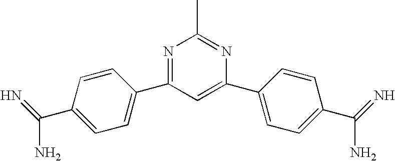

43. A compound having the general formula: 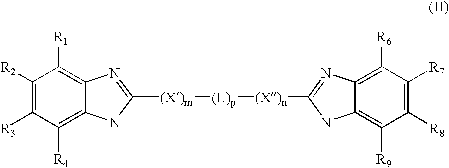
wherein:
m is an integer from 0 to 5;
n is an integer from 0 to 5;
p is an integer from 0 to 5;
X′ and X″ are each independently phenyl or thiophene;
L is selected from the group consisting of C₁₋₁₀ straight chain alkyl, C₁₋₁₀ branched chain alkyl, cycloalkyl, phenyl, naphthyl, and alkyl-substituted phenyl;
R₁, R₂, R₃, R₄, R₅, R₆, R₇, R₈, and R₉ are each independently selected from the group consisting of H, alkyl, hydroxyl, alkyloxy, oxyalkyl, halo, aryl, and Y, wherein at least one of R₁, R₂, R₃, R₄, R₅, R₆, R₇, R₈, and R₉ is Y, and Y is selected from the group consisting of: 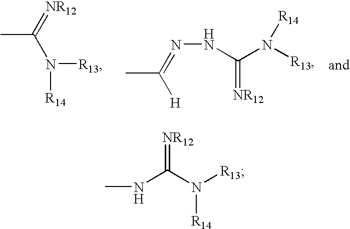
wherein:
R₁₂ is selected from the group consisting of H, hydroxyl, cycloalkyl, aryl, aralkyl, alkoxyl, hydroxycycloalkyl, alkoxycycloalkyl, hydroxyalkyl, aminoalkyl, acyloxyl, and alkylaminoalkyl;
R₁₃ and R₁₄ are each independently selected from the group consisting of H, hydroxyl, alkyl, alkoxyalkyl, cycloalkyl, aryl, aralkyl, hydroxyalkyl, aminoalkyl, and alkylaminoalkyl;
or R₁₂ and R₁₃ together represent a C₂ to C₁₀ alkyl, hydroxyalkyl, or alkylene;
or R₁₂ and R₁₃ together are: 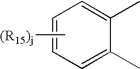
wherein:
j is an integer from 1 to 3, and R₁₅ is H or Y, as set forth above.

44. The compound according to claim 43, wherein:
p is 0;
m and n are each 1;
X′ and X″ are each phenyl; and
R₃ and R₈ are each

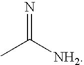

45. The compound according to claim 44, wherein the compound has the following structure:

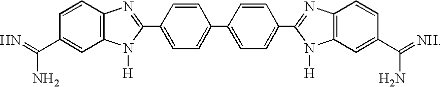

46. The compound according to claim 43, wherein,
m and n are each 0;
p is 1;
L is naphthyl; and
R₃ and R₈ are each

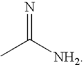

47. The compound according to claim 46, wherein the compound has the following structure:

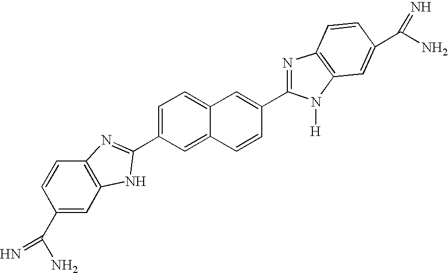

48. The compound according to claim 43, wherein,
m and n are each 1;
p is 2;
X′ and X″ are each phenyl;
L is alkyl; and
R₃ and R₈ are each

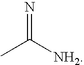

49. The compound according to claim 48, wherein the compound has the following structure:

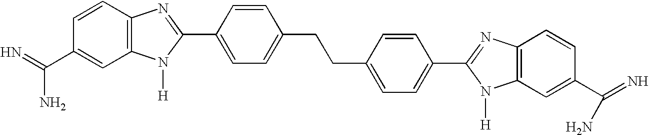

50. The compound according to claim 43, wherein:
m, n, and p are each 1;
X′ and X″ are each phenyl;
L is alkyl; and
R₃ and R₈ are each

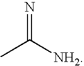

51. The compound according to claim 50, wherein the compound has the following structure:

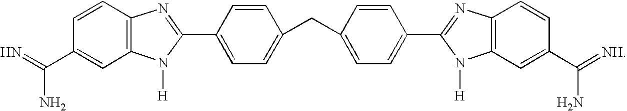

52. The compound according to claim 43, wherein:
m, n, and pare each 1;
X′ and X″ are each phenyl;
L is cycloalkyl; and
R₃ and R₈ are each

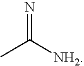

53. The compound according to claim 52, wherein the compound has the following structure:

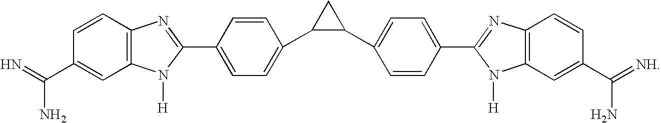

54. The compound according to claim 43, wherein:
m, n, and p are each 1;
L is alkyl;
X′ is thiophene;
X″ is phenyl; and
R₃ and R₈ are each

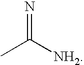

55. The compound according to claim 54, wherein the compound has the following structure:

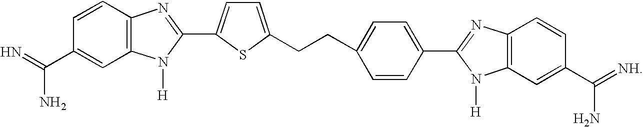

56. A compound according to claim 43, wherein:
p is 1;
m and n are each 0;
L is alkyl-substituted phenyl;
R₁, R₃, R₄, R₆, R₈, and R₉ are each hydrogen; and
R₂ and R₇ are each

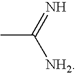

57. A compound according to claim 56, wherein the compound has the following structure:

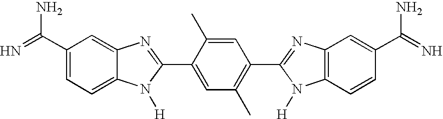

58. A compound according to claim 43, wherein:
p, m, and n are each 1;
X′ and X″ are each alkyl;
L is phenyl;
R₁, R₃, R₄, R₆, R₈, and R₉ are each hydrogen; and
R₂ and R₇ are each

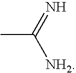

59. The compound according to claim 58, wherein the compound has the following structure:

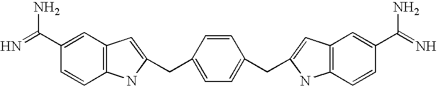

60. A compound according to claim 43, wherein:
p is 1;
m and n are each 0;
L is phenyl; and
R₃ and R₈ are

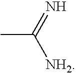

61. The compound according to claim 60, wherein the compound has the following structure:

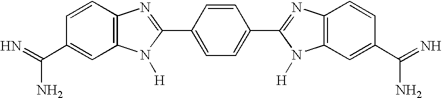

62. A compound according to claim 43, wherein:
m and n are each 1;
p is 2;
X′ and X″ are each phenyl;
L is alkyl; and
R₂ and R₇ are:

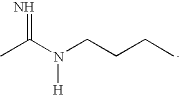

63. The compound according to claim 62, wherein the compound has the following structure:

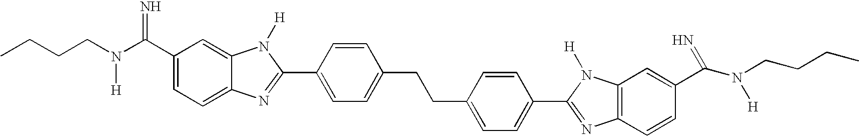

64. A compound having the general formula: 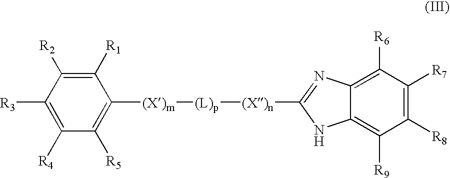
wherein:
L is phenyl, pyridine, or hydroxy-phenyl;
p, m and n are each independently an integer from 0 to 5;
X′ and X″ are each independently selected from the group consisting of C₁₋₁₀ straight chain alkyl, C₁₋₁₀ branched chain alkyl, and cycloalkyl;
R₁, R₂, R₃, R₄, R₅, R₆, R₇, R₈, and R₉ are each independently selected from the group consisting of H, alkyl, hydroxyl, alkyloxy, oxyalkyl, halo, aryl, and Y, wherein at least one of R₁, R₂, R₃, R₄, R₅, R₆, R₇, R₈, and R₉ is Y, and Y is selected from the group consisting of: 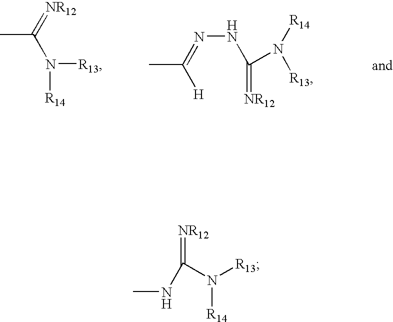
wherein:
R₁₂ is selected from the group consisting of H, hydroxyl, cycloalkyl, aryl, aralkyl, alkoxyl, hydroxycycloalkyl, alkoxycycloalkyl, hydroxyalkyl, aminoalkyl, acyloxyl, and alkylaminoalkyl;
R₁₃ and R₁₄ are each independently selected from the group consisting of H, hydroxyl, alkyl, alkoxyalkyl, cycloalkyl, aryl, aralkyl, hydroxyalkyl, aminoalkyl, and alkylaminoalkyl;
or R₁₂ and R₁₃ together represent a C₂ to C₁₀ alkyl, hydroxyalkyl, or alkylene;
or R₁₂ and R₁₃ together are: 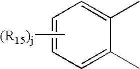
wherein:
j is an integer from 1 to 3, and R₁₅ is H or Y, as set forth above.

65. The compound according to claim 65, wherein:
n is 0;
m and p are each 1;
L is phenyl;
X′ is alkyl;
R₃ is alkoxyl; and
R₈ is

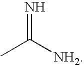

66. The compound according to claim 65, wherein the compound has the following structure:

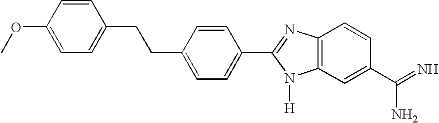

67. The compound according to claim 64, wherein:
n is 0;
m and p are each 1;
L is phenyl;
X′ is alkyl;
R₃ is alkyl; and
R₈ is

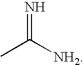

68. The compound according to claim 67, wherein the compound has the following structure:

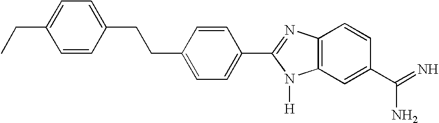

69. The compound according to claim 64, wherein:
n is 0;
m and p are each 1;
L is phenyl;
X′ is alkyl;
R₃ is halo; and
R₈ is

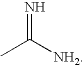

70. The compound according to claim 69, wherein the compound has the following structure:

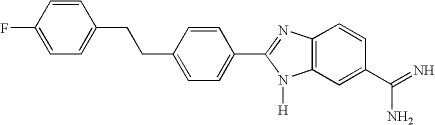

71. The compound according to claim 64, wherein;
m and n are each 0;
p is 1;
L is pyridine; and
R₃ and R₈ are each

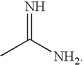

72. The compound according to claim 71, wherein the compound has the following structure:

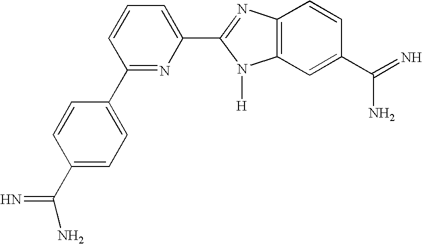

73. The compound according to claim 64, wherein:
p=1;
m and n are each 0;
L is hydroxy-phenyl; and
R₃ and R₈ are each

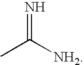

74. The compound according to claim 73, wherein the compound has the following structure:

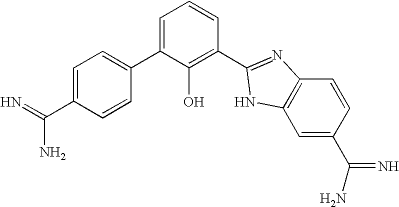

75. A compound having the general formula: 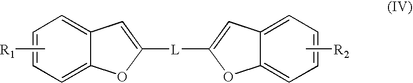
wherein L is selected from the group consisting of C₂₋₁₀ straight chain alkyl, C₁₋₁₀ branched chain alkyl, and cycloalkyl;
R₁ and R₂ are selected from the group consisting of: 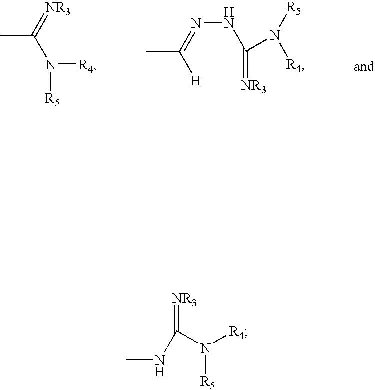
wherein R₃ is selected from the group consisting of H, hydroxyl, cycloalkyl, aryl, aralkyl, alkoxyl, hydroxycycloalkyl, alkoxycycloalkyl, hydroxyalkyl, aminoalkyl, acyloxyl, and alkylaminoalkyl;
R₄ and R₅ are each independently selected from the group consisting of H, hydroxyl, alkyl, alkoxyalkyl, cycloalkyl, aryl, aralkyl, hydroxyalkyl, aminoalkyl, and alkylaminoalkyl;
or R₃ and R₄ together represent a C₂ to C₁₀ alkyl, hydroxyalkyl, or alkylene;
or R₄ and R₅ together are: 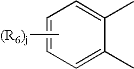
wherein:
j is a number from 1 to 3, and R₆ is selected from the group consisting of H and the groups from which R₁ and R₂ may be selected.

76. The compound according to claim 75, wherein:
L is alkyl; and
R₁ and R₂ are each

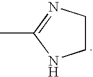

77. The compound according to claim 76, wherein the compound has the following structure:

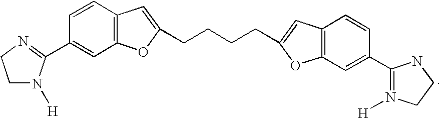

78. The compound according to claim 76, wherein:
L is alkyl; and
R₁ and R₂ are each

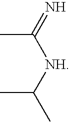

79. The compound according to claim 78, wherein the compound has the following structure:

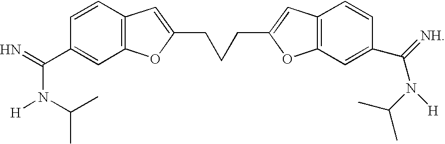

80. A compound having the general formula:

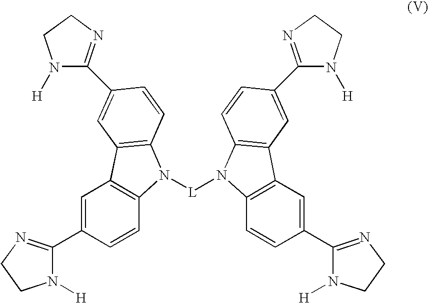

81. The compound according to claim 80, wherein L is alkyl.

82. The compound according to claim 81, wherein the compound has the following structure:

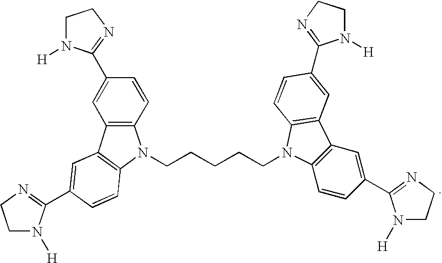

83. A compound having the general formula: 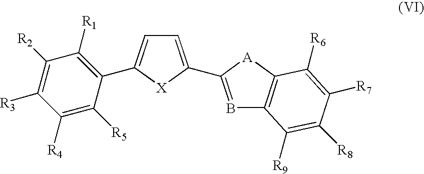
wherein:
X is oxygen;
A and B are each either nitrogen or oxygen;
R₁, R₂, R₃, R₄, R₅, R₆, R₇, R₈, and R₉ are each independently selected from the group consisting of H, alkyl, hydroxyl, alkyloxy, oxyalkyl, halo, aryl, and Y, wherein at least one of R₁, R₂, R₃, R₄, R₅, R₆, R₇, R₈, and R₉ is Y, and Y is selected from the group consisting of: 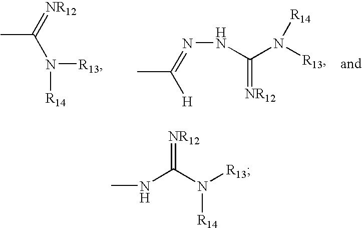
wherein:
R₁₂ is selected from the group consisting of H, hydroxyl, cycloalkyl, aryl, aralkyl, alkoxyl, hydroxycycloalkyl, alkoxycycloalkyl, hydroxyalkyl, aminoalkyl, acyloxyl, and alkylaminoalkyl;
R₁₃ and R₁₄ are each independently selected from the group consisting of H, hydroxyl, alkyl, alkoxyalkyl, cycloalkyl, aryl, aralkyl, hydroxyalkyl, aminoalkyl, and alkylaminoalkyl;
or R₁₂ and R₁₃ together represent a C₂ to C₁₀ alkyl, hydroxyalkyl, or alkylene;
or R₁₂ and R₁₃ together are: 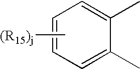
wherein:
j is an integer from 1 to 3, and R₁₅ is H or Y, as set forth above.

84. The compound according to claim 83, wherein:
X is oxygen;
A is oxygen;
B is nitrogen; and
R₃ and R₈ are each

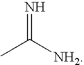

85. The compound according to claim 84, wherein the compound has the following structure:

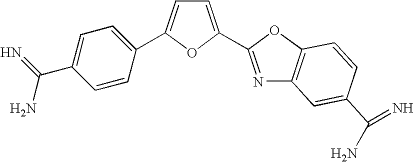

86. A compound having the general formula: 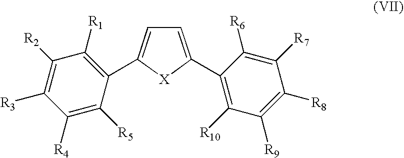
wherein:
X is oxygen; and
R₁, R₂, R₃, R₄, R₅, R₆, R₇, R₈, R₉, and R₁₀ are each independently selected from the group consisting of H, alkyl, hydroxyl, oxyalkyl, alkyloxy, alkylthio, halo, aryl, and Y, wherein at least one of R₁, R₂, R₃, R₄, R₅, R₆s R₇, R₈, R₉, and R₁₀ is Y, and Y is selected from the group consisting of: 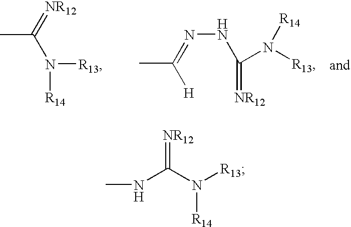
wherein:
R₁₂ is selected from the group consisting of H, hydroxyl, cycloalkyl, aryl, aralkyl, alkoxyl, hydroxycycloalkyl, alkoxycycloalkyl, hydroxyalkyl, aminoalkyl, acyloxy, and alkylaminoalkyl;
R₁₃ and R₁₄ are each independently selected from the group consisting of H, hydroxyl, alkyl, alkoxyalkyl, cycloalkyl, aryl, aralkyl, hydroxyalkyl, aminoalkyl, and alkylaminoalkyl;
or R₁₃ and R₁₄ together are: 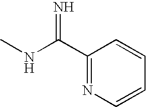
or R₁₂ and R₁₃ together represent a C₂ to C₁₀ alkyl, hydroxyalkyl, or alkylene;
or R₁₂ and R₁₃ together are: 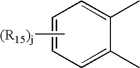
wherein:
j is an integer from 1 to 3, and R₁₅ is H or Y, as set forth above.

87. The compound according to claim 86, wherein:
X is oxygen;
R₂ and R₇ are each alkylthio; and
R₃ and R₈ are each

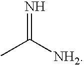

88. The compound according to claim 87, wherein the compound has the following structure:

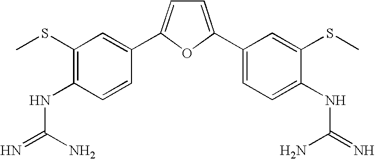

89. The compound according to claim 86, wherein:
X is oxygen;
R₁ and R₆ are hydroxyl;
and R₃ and R₈ are each:

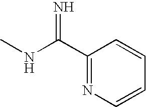

90. The compound according to claim 89, wherein the compound has the following structure: [chemical structure]
